(19) United States
(12) Patent Application Publication
(10) Pub. No.: US 2006/0148742 A1
(43) Pub. Date: Jul. 6, 2006

(54) Polynucleotide delivery to cardiac tissue

(76) Inventors: David Martin Kaye, Beaumaris (AU); John Melmouth Power, Williamstown (AU); Clifton A. Alferness, Port Orchard, WA (US); Adam Lucas Bilney, Wy Yung (AU); Kenneth Chien, Cambridge, MA (US); Athanasios Cristos Preovolos, East Kew (AU)

Correspondence Address: Michael L. Goldman;Nixon Peabody LLP, Clinton Square, P.O. Box 31051, Rochester, NY, 14603, US

(21) Appl. No.: 11/212,049

(22) Filed: Aug. 25, 2005

Related U.S. Application Data
(63) Continuation-in-part of application No. PCT/AU05/00237, filed on Feb. 23, 2005.
(60) Provisional application No. 60/548,038, filed on Feb. 26, 2004.
(60) Provisional application No. 60/612,846, filed on Sep. 24, 2004.

(51) Int. Cl. A61K 48/00

(52) U.S. Cl. 514/44

(57) ABSTRACT

A method for delivering a polynucleotide to cardiac tissue, including substantially isolating the coronary venous circulation from systemic circulation, and introducing a polynucleotide into the isolated coronary venous circulation to effect localized transfection of cardiac tissue. The polynucleotide advantageously produces a therapeutic effect, such as increasing or decreasing the expression level of a protein in the cardiac tissue.

This application is a continuation-in-part of PCT/AU2005/000237, filed month/day/year, which is hereby incorporated by reference in its entirety.

### FIELD OF THE INVENTION

The present invention relates to the field of cardiology and more specifically to the delivery of therapeutic polynucleotides to cardiac tissue.

### BACKGROUND TO THE INVENTION

Heart disease is a major public health issue of very high prevalence, especially in the Western world. Cardiac conditions include coronary artery disease, ischaemic heart disease, heart failure, valvular heart disease, cardiac arrhythmias and cardiac inflammation (myocarditis) to name a few. Coronary artery disease and heart failure are possibly the most serious and prevalent, together being a leading cause of death in the Western world. The impact of acute myocardial infarction and congestive heart failure and their sequelae on the quality of life of patients and the cost of health care drives the search for new therapies.

Congestive heart failure (CHF) is a serious condition in which the heart loses its ability to pump blood efficiently. Data from the National Heart, Lung and Blood Institute, suggests about 5 million people in the United States alone have heart failure, and another 550,000 new cases are diagnosed each year. CHF contributes to or causes about 300,000 deaths annually. The disease is most common in people aged 65 or older, women and African Americans. The most common symptoms of heart failure are shortness of breath, feeling tired, and swelling in the ankles, feet, legs, and sometimes the abdomen. There is no cure for congestive heart failure, and a clear need exists in the art for effective therapies.

While there is continual discovery of new and efficacious compounds to treat heart disease, delivery of the therapeutically active agents to cardiac tissue can be problematic. For example, the structure of many pharmaceuticals may be altered by the liver, destroying their therapeutic activity. Accordingly, systemic administration (i.e. by oral, IV, IM routes and the like) is often sub optimal. This problem has been overcome in part by using sublingual or rectal administration to avoid “first pass” degradation through the liver. However, after these routes of administration the drug can still be degraded on subsequent passes through the liver.

Another problem relates to toxicity of therapeutic agents. For example, a drug administered to target a tumor of the heart may have a toxic effect on healthy tissue in other parts of the body. Indeed, anticancer treatments are often discontinued due to toxicity problems, frequently leading to further progression of the cancer.

Another problem in the delivery of therapeutic agents to tissues of the heart arises where agents intended for treatment of the heart alone are lost to the systemic circulation where they are metabolized without benefit, or have a deleterious effect on other healthy tissues. In some cases, significant amounts of the therapeutic agent may be needed, and efficiency of the treatment is therefore reduced by loss of the agent to the general circulation and time of exposure to the heart tissue. This may be a significant issue in the introduction of therapeutic nucleic acid molecules to the heart. It is desirable to maximize the modification of the targeted organ, tissue, or cell by a therapeutic nucleic acid to the target, such that the targeted organ, tissue or cell has a higher level of exposure to the therapeutic nucleic acid than the non-targeted organ tissue or cell. Such preferential exposure can be important since modification by a therapeutic nucleic acid may be irreversible.

In United States Patent Application 20020062121 (Tryggvason et al.), there is exemplified a method for the delivery of therapeutic agents to epithelial cells of the liver and lung of a mammal utilizing a closed perfusion system. While this document demonstrates delivery to organs having a comparatively simple vasculature, the document fails to disclose methods useful for delivering therapeutics to more complex organs.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### IN THE FIGURES

FIG. 1 is a simplified illustration of human heart showing a number of the cardiac veins and arteries which make up the coronary circulation.

FIG. 2 is a flow diagram illustrating steps performed in a method of isolating the coronary venous circulation according to an embodiment of the present invention.

FIG. 3A illustrates a venous collection device having a support structure in the form of a frame for use with an embodiment of the invention.

FIG. 3B illustrates an alternative embodiment of a venous collection device having a support structure which is a variation of the support structure illustrated in FIG. 3A.

FIG. 3C illustrates another embodiment of a venous collection device incorporating a woven support structure.

FIG. 3D illustrates yet another embodiment of a venous collection device incorporating a flange.

FIG. 3E illustrates still another embodiment of a venous collection device having a support structure which incorporates an inflatable balloon.

FIG. 3F is a cross section view of the support structure of FIG. 3E.

FIG. 3G illustrates an alternative embodiment of a venous collection device having a support structure which also incorporates an inflatable balloon.

FIG. 4A illustrates a delivery device according to an embodiment of the invention showing an un-inflated occluding means.

FIG. 4B illustrates the delivery device of FIG. 4A with the occluding means inflated.

FIG. 5 shows a re-circulation model according to an embodiment of the present invention.

FIG. 6 illustrates the results of treatment of sheep with pacing induced heart failure using an embodiment of the present invention and either adenovirus encoding the sarcoplasmic reticulum ATPase 2a or a control gene for β-Galactosidase.

FIG. 7 shows immunohistochemical staining for β-Galactosidase in heart indicating high efficiency transduction of the majority of cardiomyocytes using an embodiment of the invention with adeno-associated virus encoding the gene for β-Galactosidase (LacZ) [AAV2/LacZ].

FIG. 8 shows a map of genomic AAV serotype 2/1 demonstrating the position of insertion of SERCA2a polynucleotide.

FIG. 9 shows the selective delivery of SERCA2a to cardiac tissue using the present methods. Lanes 1 and 2, positive control from HEK cells infected with AdSERCA2a (5 μg and 10 μg respectively); Lane 3 blank; Lanes 4 to 6 cells from an animal having chronic heart failure after administration of AdSERCA2a, Lanes 4 and 5 heart (5 μg and 10 μg respectively), Lane 6 liver (10 μg), Lanes 7 and 8 cells from an animal having chronic heart failure after sham administration of AdSERCA2a, Lane 7 heart (10 μg), Lane 8 liver (10 μg). The position of the SERCA2a band is indicated by the arrow.

FIG. 10 shows improvement of LVEF (x-axis) as a function of days post administration of AdSERCA2a (y-axis) in animals treated by the protocol described in Example 6 herein. The first three bars relate to sham-treated animals (p=0.03), with the remaining bars relating to animals administered SERCA2a (p=0.02).

### SUMMARY OF THE INVENTION

Briefly, in one aspect the present invention provides a method for delivering a polynucleotide to cardiac tissue. Using the method, the output from the coronary veins, i.e. the coronary venous circulation, is substantially, or completely isolated from the systemic circulation. As a result, a polynucleotide introduced into the coronary arterial circulation is thereby selectively delivered to the heart tissue. The polynucleotide may or may not be recirculated from the coronary venous circulation to the coronary arterial circulation. Preferably, the polynucleotide is recirculated.

Advantageously, substantial isolation of the coronary venous circulation from the systemic circulation provides for the preferential delivery of a polynucleotide to cardiac tissue, while exposure of non-cardiac tissue to the polynucleotide is minimized.

Preferably, the coronary venous circulation is isolated from the systemic circulation by occluding the flow between the coronary sinus and the systemic circulation. Any polynucleotide exiting the coronary venous circulation is therefore restricted from entering the systemic circulation (e.g. the vena cavae or right atrium) for transport to other parts of the body.

The method may include use of a venous collection device in the coronary sinus to drain polynucleotide from the cardiac tissue. The venous collection device may include a support to maintain patency of the coronary sinus during collection of fluid therefrom. An artificial flow path is established between the venous collection device and the one or more coronary arteries and the polynucleotide is added to the artificial flow path for delivery to the heart.

Preferably, the support structure comprises a two- or three-dimensional framework which is deliverable to the coronary sinus in a compressed state. The framework is expandable upon release of the compressed structure from a delivery lumen to maintain patency within the coronary sinus. The support structure for maintaining patency of the coronary sinus during collection of fluid therefrom is preferably percutaneously deliverable.

In another embodiment, the support structure may comprise two consecutively inflatable regions. The first region is configured to, when inflated, rest in abutment with a portion of the right atrium wall surrounding the coronary sinus ostium. The second region is configured to, when inflated, maintain patency of the coronary sinus while flow between the coronary sinus and the right atrium is occluded. These balloon regions may be used with or without a compressible support structure such as the framework described above.

Embodiments of the present invention may be used for the delivery of a substance to the substantially isolated coronary venous circulation. Any of the embodiments of the inventive methods may further include the step of introducing a substance to fluid in the artificial flow path of the substantially isolated coronary venous circulation. In one embodiment, the substance is a therapeutic substance selected from the group consisting of one or more of a virus, a pharmaceutical, a peptide, a hormone, a stem cell, a cytokine, an enzyme, a polynucleotide, a polynucleotide delivery agent, blood and blood serum.

In one embodiment, the polynucleotide decreases the expression of a nucleic acid sequence within a cell of the cardiovascular system; in other embodiments the polynucleotide initiates or increases the production of a protein within a cell of the cardiovascular system; and in others, more than one nucleic acid or protein is increased and/or decreased through the use of one or more polynucleotides simultaneously or in sequence. In some embodiments of this aspect of the invention the decrease and/or increase of the targeted nucleic acid(s) and/or protein(s) within a cell of the cardiovascular system treats a disease of the cardiovascular system.

The disease of the cardiovascular system being treated may include, but is not limited to a disease selected from the group consisting of heart failure, ischemia, arrhythmia, myocardial infarction, congestive heart failure, transplant rejection, abnormal heart contractility, non-ischaemic cardiomyopathy, mitral valve regurgitation, refractory myocardial ischaemia, non-ischaemic heart failure, aortic stenosis or regurgitation, and abnormal Ca²⁺ metabolism. In one embodiment, the increase and/or decrease of the targeted protein or nucleic acid is sustained, e.g., for at least about one, two, three, four, five, six, or more months. In other embodiments, the effect if sustained for at least about one, two, three, four, five, six, or more years.

In a further aspect of any of the embodiments of the invention the polynucleotide is delivered to a cell in a viral vector, a non-viral vector, or one or more polynucleotides are delivered in a combination of viral and/or non-viral vectors. Viral vectors useful in embodiments of the invention include, but are not limited to viral vectors selected from the group consisting of an adenovirus, a retrovirus, a herpes simplex virus, a bovine papilloma virus, an adeno-associated virus, a lentiviral vector, a vaccinia virus, and a polyoma virus. Non-viral vectors useful in embodiments of the invention include, but are not limited to non-viral vectors selected from the group consisting of direct delivery of DNA such as by perfusion, injection, naked DNA delivery, liposome mediated transfection, encapsulation, receptor-mediated endocytosis, and microprojectile bombardment. In a further embodiment of the current invention, the viral vector is an adenovirus or an adeno-associated virus.

A further aspect of any of the embodiments of the invention including a viral vector, comprises the addition of less than about 1×10¹³, 1×10¹², 1×10¹¹, 1×10¹⁰, 1×10⁹, or 1×10⁸ viral particles to the fluid in the artificial flow path; and/or that the viral vector transfects at least equal to, or about, 10%, 20%, 30%, 40%, 50%, 60%, 70%, 80%, 90%, 95%, 99%, or 100% of cardiomyocytes of the targeted vascular territory. The number of viral vector can also be a therapeutically effective amount, such that a sufficient amount of polynucleotide is delivered to the cardiac tissue to effect a therapeutic result.

Another aspect of any of the embodiments of the invention including a polynucleotide that improves or sustains an ejection fraction of the cardiovascular system. The ejection fraction can be improved by, for example, at least 5%, 10%, or 15%. In a preferred embodiment the ejection fraction is improved by 20% or greater, e.g. 25%, 30%, 35%, 40%, 45%, 50%, 55%, 60%, 70%, 75%, 80%, 85%, 90%, 95%, 100%, or more. Typically, the ejection fraction will be improved or sustained by the present methods, however it should be understood that the polynucleotide may still be considered to have a therapeutic effect if a marked decrease in the rate of degeneration of the ejection fraction was noted after commencement of therapy.

It will be understood that the present method may be used to interfere with the natural state of a cardiac cell by a number of mechanisms. In one aspect the method leads to an increase in production of a protein that positively affects cardiac function, such sarcoplasmic reticulum ATPase (SERCA). Alternatively, the method may include a mechanism for the down-regulation of a protein that negatively affects cardiac function such as phospholamban. Yet another strategy to interfere with the natural state of the cardiac cell is to prevent a protein/protein interaction of the cell. An example of this approach is where the interaction between the proteins SERCA and phospholamban is inhibited.

Down-regulating levels of proteins detrimental to cardiac function such as phospholamban may be achieved using techniques such as antisense inhibitor molecules capable of hybridizing to specific mRNA sequences to form a duplex structure. The bound inhibitor interferes with the normal function of mRNA as a template for protein translation.

Another mechanism of down-regulating protein production involves the use of siRNA and RNAi methods whereby an antisense molecule hybridizes to targets a specific mRNA sequence to form a duplex structure, the duplex being vulnerable to cleavage by double-stranded RNase molecules. The cleaved mRNA molecule is able to produce only a truncated protein that has a decreased biological activity.

Protein regulation may also be up-regulated or down-regulated at the transcriptional level by enhancer or repressor molecules. For example, it has been shown that transcription of phospholamban can be inhibited by the use of a zinc finger protein (ZFP) transcriptional repressor. It has been demonstrated that a ZFP targeting the phospholamban promoter significantly improved several cardiac parameters in a rat model of congestive heart failure (Zhang et al., 2005 Annual Meeting of American Society of Gene Therapy, St. Louis, Abstract #43).

A further aspect of the invention features a method of treating a subject, suffering from, or at risk of cardiovascular disease, e.g., a subject having heart failure. The method includes introducing a polynucleotide into the substantially isolated coronary venous circulation of the subject. A delivery system, such as, a viral vector can be used to administer the polynucleotide, as a therapeutic nucleic acid. In one embodiment, the subject is a human. In another embodiment, the subject is a non-human mammal, a bird, a fish, or an amphibian.

In one embodiment, the viral delivery system is an adenoviral or lentiviral delivery system. In another embodiment, the viral delivery system is an. adeno-associated viral delivery system. The adeno-associated virus may advantageously be of serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9). However, in a preferred form of the invention the adeno-associated virus is of serotype 1, and is more preferably AAV2/1 (i.e. containing the capsid sequences of AAV1 and the ITRs from AAV2).

In one embodiment, the subject has or is at risk of heart failure, e.g. a non-ischemic cardiomyopathy, mitral valve regurgitation, ischemic cardiomyopathy, or aortic stenosis or regurgitation.

In another embodiment, the polynucleotide encodes a protein, e.g., a heart-specific protein or a protein effective in modulating cardiac physiology. In a further embodiment, the expression of the protein encoded by the polynucleotide is sustained, e.g., for at least one, two, three or more months.

In one embodiment, the polynucleotide encodes a protein involved in the regulation of calcium cycling in cardiomyocytes, such as a sarcoplasmic endoplasmic reticulum Ca²⁺ ATPase pump. The sarcoplasmic reticulum is integral to cardiac excitation-contraction coupling. There is re-uptake of calcium into the sarcoplasmic reticulum by sarcoplasmic reticulum/endoplasmic reticulum calcium ATPase (SERCA). Decreased SERCA2a (i.e., the 2a isoform of SERCA) activity is a common feature of cardiomyopathy. Thus, the present methods may be used to advantageously augment the heart's endogenous supply of SERCA2a.

In another form of the invention the polynucleotide encodes a protein capable of indirectly modulating cardiac physiology. As discussed supra, the cardiac protein phospholamban is inhibitory to the activity of SERCA2a. Accordingly, the present methods include methods to decrease the level or activity of phospholamban in a cardiac cell. In a preferred embodiment expression of a pseudophosphorylated mutant of phospholamban is increased. A preferred mutant has replacement of the serine 16 phosphorylation site with the basic amino acid glutamine, thereby introducing a negative charge at position 16 (S16E phospholamban mutant). This pseudophosphorylated form of phospholamban competes with natural phospholamban for binding to SERCA, thereby decreasing the opportunity for the natural protein to negatively affect SERCA activity.

In a preferred form of the method the expression of the polynucleotide is sustained for at least about one, two, three, four, five, six months, or longer.

In one embodiment, and where the polynucleotide is delivered by viral vector, the number of viral particles (vp) or viral genomes (vg) introduced to the substantially isolated coronary venous circulation is less than or equal to about 1×10⁸, 1×10⁹, 1×10¹, 1×10¹¹, 1×10¹², or 1×10¹³. The amount is advantageously sufficient to result in a therapeutic effect, i.e., to treat or prevent or inhibit the progression of one or more characteristics of a disease.

In one embodiment, treating the subject ameliorates at least one symptom of heart failure. Therapeutic effect on heart failure symptoms can be evaluated in the subject by determining for example: survival, cardiac metabolism, heart contractility, heart rate, ventricular function, e.g., left ventricular end-diastolic pressure (LVEDP), left ventricular systolic pressure (LVSP), end systolic pressure volume relationship (ESPVR), Ca²⁺ metabolism, e.g., intracellular Ca²⁺ concentration, e.g., peak or resting [Ca²]. The evaluation can be performed before, after, or during the treatment.

In a preferred embodiment, the subject, e.g., a human or a non-human animal, is at risk for, or has, a heart disorder, e.g., heart failure, ischemia, arrhythmia, myocardial infarction, congestive heart failure, transplant rejection, abnormal heart contractility, abnormal Ca²⁺ metabolism, or congenital heart disease.

In one embodiment, the heart disorder is heart failure, ischemia, arrhythmia, myocardial infarction, congestive heart failure, transplant rejection, abnormal heart contractility, abnormal Ca²⁺ metabolism, or congenital heart disease.

In one form of the method, a polynucleotide is introduced into the arterial circulation of an animal where the coronary venous circulation is substantially isolated from the systemic circulation such that the polynucleotide is substantially prevented from entering the systemic circulation. In a preferred embodiment, where the animal has congestive heart failure the polynucleotide is capable of expressing a SERCA protein. In a further preferred form of this embodiment, the level of expression of SERCA in at least some cardiac cells of the animal is increased as a result of introducing the polynucleotide encoding SERCA. In yet another form of this embodiment, the expression of SERCA in at least some cardiac cells of the animal is increased at least about 1.5 to two-fold. In another form of this embodiment, where the animal has congestive heart failure, a LVEF of the animal is higher than that of the animal prior to exposure to the exogenous polynucleotide encoding SERCA. In a further preferred embodiment, the LVEF of the animal is about 1.6-fold higher. The assessment of the level of expression of SERCA or the LVEF can be made at one or more times after introduction of the polynucleotide, such as about 1, 2, 3, 4, 5, 6 days, 1, 2, 3, 4 weeks, 1, 2, 3, 4, 5, 6, 7, 8, 9, 10, 11, months or 1, 2, 3, 4, 5, 6, 7, 8, 9, 10, or more years.

Also provided by the present invention is a method for treating or preventing a heart condition including heart failure, ischemia, arrhythmia, myocardial infarction, congestive heart failure, transplant rejection, abnormal heart contractility, non-ischemic cardiomyopathy, mitral valve regurgitation, aortic stenosis or regurgitation, and abnormal Ca²⁺ metabolism including use of a method described herein. Also provided is the use of a polynucleotide or vector as described herein in the treatment or prevention of a disease of the cardiovascular system.

In another embodiment, the nucleic acids are introduced into the subject by somatic gene transfer and are not introduced into the germ line of the subject.

### DETAILED DESCRIPTION OF THE INVENTION

Delivering therapeutic agents to the heart for treatment of the heart tissue is more complicated than delivery to other organs because the heart must generate cardiac output as well as provide its own blood supply. Isolation of the heart's own blood supply from the systemic circulation is therefore desirable but challenging because of the potential variation between patients in cardiac vasculature, and the complex topography which has in the past deterred attempts at isolation. Furthermore, given that the heart supplies blood to all tissues of the body, there is a significant risk that the delivery of any therapeutic agent to the heart will result in delivery to non-cardiac cells.

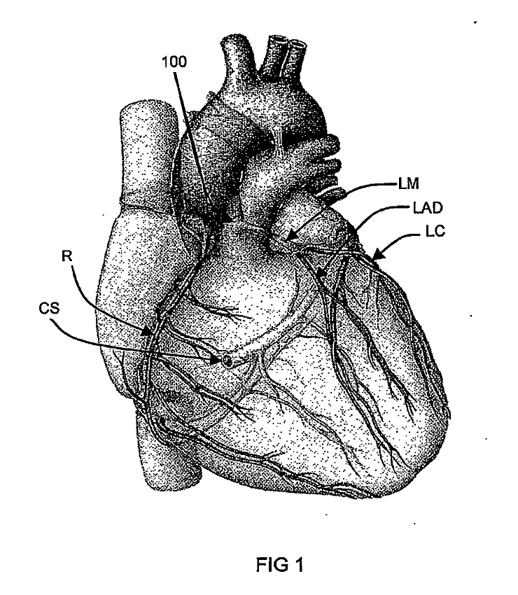

Normally, four main coronary arteries provide oxygenated blood to the heart for distribution throughout the heart tissue; the left anterior descending (LAD), left circumflex (LC), left main (LM) and right (R) coronary arteries. These are shown in the illustration of the heart provided in FIG. 1. The coronary ostia 102 opening into these arteries are generally found in the aortic sinus, just above the cusps of the aortic valve, below the sinotubular junction. However, in a number of patients additional ostia are found in this region which open into accessory conal branches. These accessory ostia are usually smaller in diameter and irregularly located and are therefore difficult to isolate or catheterize using traditional approaches. Thus, in one form of the method only the left and/or right coronary arteries are cannulated. However, it is to be understood that use of other coronary arteries is not excluded, especially where they are of sufficient size. For example, applicants have demonstrated successful perfusion using only the left coronary artery.

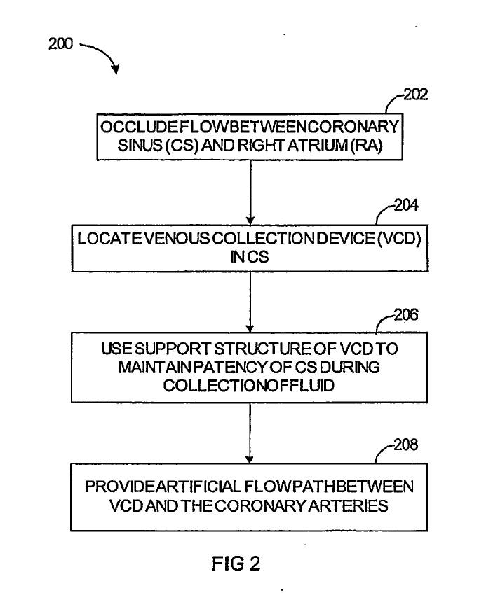

Referring now to FIG. 2, a flow diagram shows steps involved in one embodiment of the method, generally referred to at 200, for substantially isolating the coronary venous circulation from the systemic circulation. In a first step 202, flow between the coronary sinus (referred to as CS in FIG. 1) and the right atrium is occluded. In a second step 204, a venous collection device having a collection lumen and a support structure is located in the coronary sinus. In a third step 206, the support structure is used to maintain patency of the coronary sinus during collection of fluid through the collection lumen. In another step 208, an artificial flow path is provided between the coronary sinus and the one or more coronary arteries. This method makes cardiac pumping for maintenance of systemic circulation achievable while the coronary venous circulation is substantially isolated.

The steps of occluding flow between the coronary sinus and the right atrium and positioning the venous collection device in the coronary sinus may be performed in sequence or substantially simultaneously. Where these steps are performed substantially simultaneously, the venous collection device may also be an occluding device.

During isolation of the coronary venous circulation from the systemic circulation using the inventive method, it is preferable to maintain continuous circulation of fluid in the artificial flow path and in the cardiac circulation. Flow may be maintained to ensure delivery of blood (carrying oxygen and nutrients) to the cardiac tissue, and adequate delivery of therapeutic agents such as a polynucleotide, if they are being administered.

In other organs, maintenance of desired flow rates during shunting of blood (e.g. in kidney dialysis) is relatively straightforward because the vessels through which access is gained are stiff and can withstand (a) insertion of collection catheters through the vessel wall, (b) occasional contact of the collection catheter tip with the vessel wall, and (c) negative pressures generated at the catheter tip during collection of fluid. In these cases, vessel collapse resulting from contact between the catheter tip and the vessel wall is rare, and not therefore of great concern during shunting procedures.

In contrast, the coronary sinus is significantly more difficult to deal with when attempting to collect fluid using a collection lumen located within the sinus. This is in part due to the fact that the coronary sinus wall is soft and conformable, unlike stiff artery walls, and is therefore prone to collapse when contacted by a collection catheter tip. This problem is intensified as a result of the negative pressures which are generated at the catheter tip as fluid is drawn out of the sinus.

Further, because of the curvature of the coronary sinus, there is a natural tendency for a catheter tip approaching from the right atrium to contact the sinus wall, thus increasing the risk of collapse. Collapsing of the coronary sinus can cause venous pooling in the coronary veins and may therefore be fatal. Use of a venous collection device support structure to maintain patency of the coronary sinus, in accordance with the present invention, therefore minimizes the risk of these complications eventuating.

The venous collection device includes a collection lumen, an occluding body and a support structure which is configured to maintain patency of the coronary sinus. Advantageously, the support structure also maintains the tip of the collection lumen substantially centrally, relative to the walls of the coronary sinus thereby further reducing the risk of the tip contacting the vessel wall. This is particularly important because, as briefly mentioned, a small negative pressure is established at the catheter tip as venous blood is drawn out of the sinus through the collection lumen. This negative pressure increases the risk of damaging the wall of the coronary sinus if contacted by the tip and also increases the risk of the collection lumen being occluded (i.e. by the vessel wall). Centralizing the collection lumen using the support structure significantly reduces the risk of invagination of the tip of collection lumen into the coronary sinus wall.

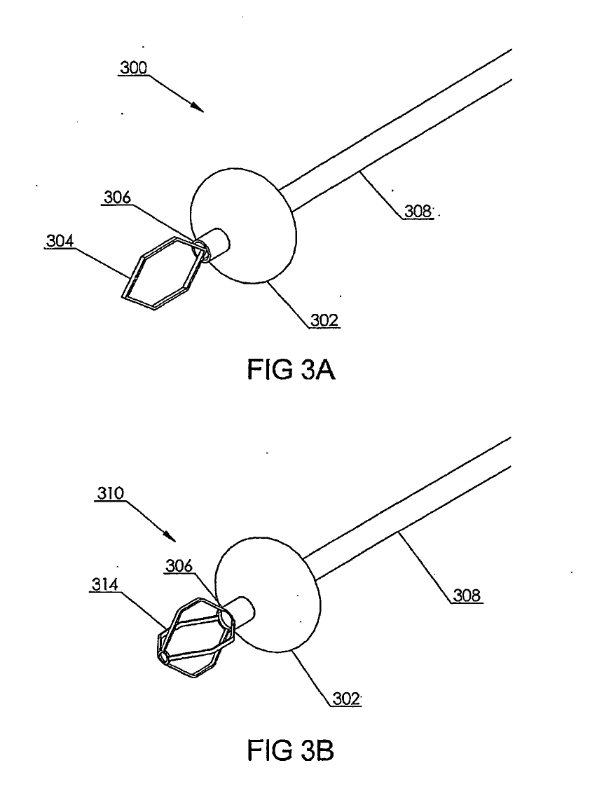

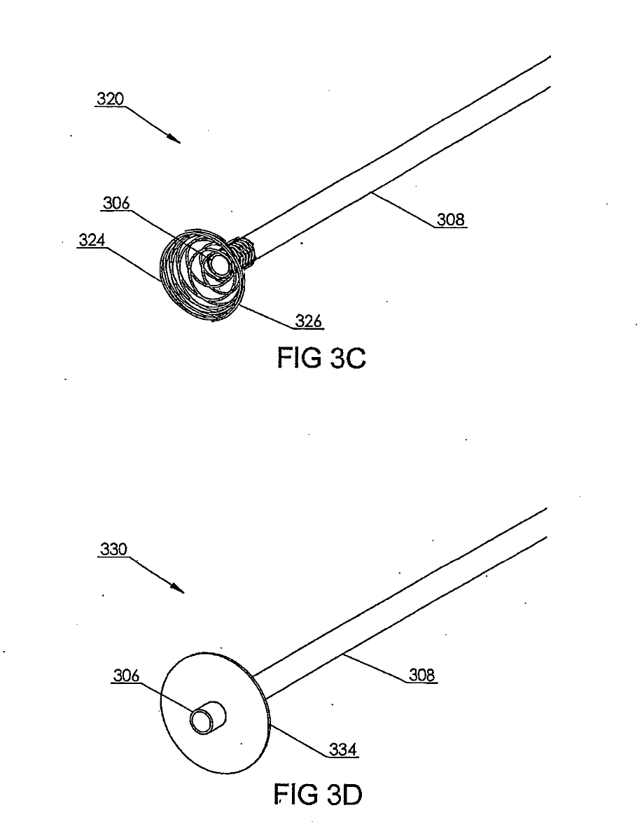

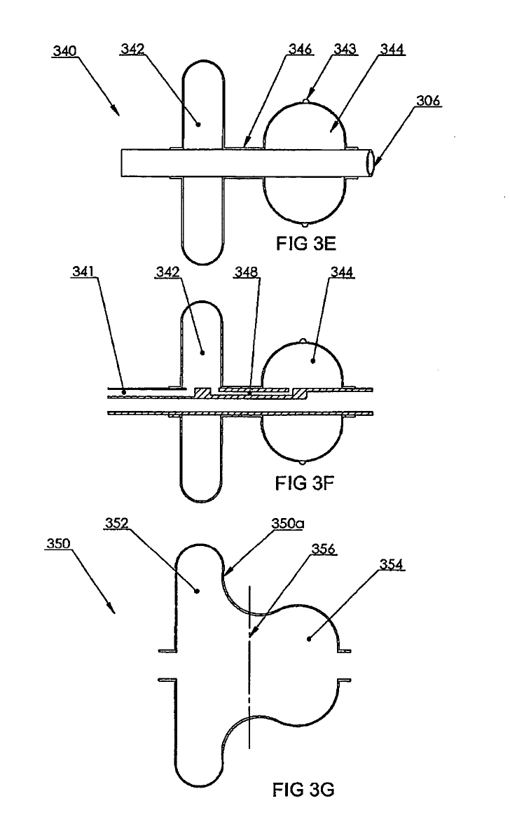

The venous collection device may be embodied in many different forms. FIGS. 3A to 3G illustrate some examples of different embodiments 300, 310 320, 330, 340 and 350 of a venous collection device. Preferably, the tip of the venous collection device is positioned just inside the coronary sinus ostium to ensure that venous blood is collected from all or at least most of the coronary veins draining into the coronary sinus.

FIG. 3A shows a venous collection device 300 having an occluding portion in the form of inflatable balloon 302 and a support structure in the form of a two-dimensional frame 304. Frame 304 is designed to compress or fold down so that it can be delivered percutaneously, and expand when released in position in the coronary sinus. When expanded, the frame preferably contacts opposing walls of the coronary sinus, thereby centralizing the tip 306 of the collection lumen 308 within the sinus. Venous blood is then collected from the sinus through collection lumen 308 and channeled into the artificial flow path.

The venous collection device 310 of FIG. 3B substantially replicates the support structure illustrated in FIG. 3A with the exception that the support structure of FIG. 3B is a three-dimensional frame 314 which opens to support the vessel wall in width and in height. Frame 314 is also designed to compress or fold down for percutaneous delivery. It is to be understood that expansion of the folded frame may be provided in a number of ways. In the embodiment illustrated, frame 314 is provided by two substantially hexagonal frame pieces arranged at right angles in such a way that they are compressible or collapsible for percutaneous delivery, and then open out to a “supporting” configuration when positioned within the coronary sinus. Other support structures may be sprung, hinged or use other suitable compression, folding and expansion mechanisms to achieve the desired functionality. Frame segments may incorporate a range of different geometrical configurations, including oblong, oval and the like, if a frame arrangement is adopted

In the embodiments illustrated in FIG. 3A and FIG. 3B, support structure 304 or 314 may be manipulated using a guide wire passing through collection lumen 308. To deploy venous collection device 300 or 310, collection lumen 308 is delivered percutaneously and tip 306 is positioned just inside the coronary sinus. When tip 306 is in place (as determined by any suitable imaging technique), balloon 302 is inflated to occlude flow between the coronary sinus and right atrium. Support structure 304 or 314 is compressed or folded so as to fit inside collection lumen 308. The compressed support structure is delivered, inside collection lumen 308 and through the opening at tip 306 to the coronary sinus. As support structure 304 or 314 s pushed out of the collection lumen through the opening at tip 306, it expands to its “supporting configuration”, thereby maintaining patency of the vessel and centralizing tip 306 of collection lumen 308 relative to the sinus walls. It is to be understood that full support of the coronary sinus walls by the support structure may not be necessary to support the sinus walls for the entire time the support structure is resident in the coronary sinus. For example, the support structure may be structured such that when expanded only one side of the support structure may contact the sinus wall. Alternatively in some situations it may be preferable to use a support structure having smaller dimensions than the sinus. In that instance, the support structure may only contact the sinus wall when the sinus wall collapses. This approach of using a support structure of smaller dimension may be less traumatic to the delicate structure of the coronary sinus.

It is to be understood that the support structure may be attached to or delivered within the collection lumen (as described herein), or it may be provided as a separate component deliverable through a delivery catheter or sheath which may also deliver or position the collection lumen and/or occluding balloon. In such an embodiment, once the support structure is positioned in the coronary sinus, the delivery catheter/sheath is retracted relative to the support structure enabling it to expand to maintain patency. Centralization of the collection lumen tip is also achieved. The delivery catheter is then removed from the patient.

The venous collection device 320 illustrated in FIG. 3C takes a different form. In this embodiment, the support structure 324 is an expandable framework having a woven or braided, basket-like configuration when expanded. In place of the inflatable occluding balloon 302, support structure 324 also has a thin silicon or other flow-proof coating 326 on the inner and/or outer surface of the framework to prevent flow between the coronary sinus and the right atrium. In a manner similar to the embodiments illustrated in FIGS. 3A and 3B, the embodiment illustrated in FIG. 3C maintains patency of the coronary sinus and centralizes the tip 306 of collection lumen 308.

The support structure 324 can be compressed or minimized for percutaneous delivery to the coronary sinus via a delivery catheter (not shown), as is the case for the devices illustrated in FIGS. 3A and 3B. This may be achieved by elongating the braid so as to reduce the diameter of the device. When in position, the delivery catheter is retracted and the support structure 324 expands to contact the inner wall of the coronary sinus, holding the sinus open and centralizing the tip 306 of the collection lumen 308 with respect to the vessel wall.

Guide wires or other ancillary fibers/devices may aid in the positioning and expansion of the support structure. When the support structure is expanded, the silicon sheath or other flow-proof coating becomes taught, occluding flow between the sinus and the right atrium. Preferably, the rim of support structure 324 has a soft coating to minimize damage to the sinus wall. It is to be understood that the support structure 324 of FIG. 3C is only one example embodiment and that other forms may be adopted, such as telescopically and radially expandable frameworks.

Preferably, the support structures 304, 314 and 324 of FIGS. 3A, 3B and 3C respectively are formed from a biocompatible shape memory material. Such materials include nitinol, a shape memory alloy. Nitinol devices can be manufactured in such a way that they can be compressed for percutaneous delivery to a deployment site and then resume a “shape memory” configuration when released from the delivery lumen into the temperate environment of the blood vessel. It is to be understood, however, that other biocompatible materials including plastics (e.g. hydrophilic plastics), ceramics and the like may also be suitable.

To minimize the risk of blocking small veins which feed into the coronary sinus, the venous collection device should sit just inside the coronary sinus ostium, or be pressed against the ostium from the right atrium chamber. In either case, a hemodynamic seal is established. It is also desirable for the collection lumen 308 to be flexible for ease of delivery and positioning within the coronary sinus and to prevent vessel tenting.

The venous collection device 330 illustrated in FIG. 3D takes yet another form. Here, occlusion is achieved by positioning flange 334 over the coronary sinus ostium, closing it from the atrial side. In this embodiment, the venous collection device should be selected with a flange diameter which is sufficient to block flow through the coronary sinus ostium when it is in abutment with the surrounding portion of the right atrium wall. Tip 306 of the collection lumen 308 sits just inside the coronary sinus, protruding centrally of the flange 334.

Advantageously, the negative pressure generated within the sinus during collection of venous flow creates a more effective seal between the flange 334 and the coronary sinus ostia, substantially restricting or preferably preventing flow from the isolated coronary venous circulation into the systemic circulation via the right atrium. To achieve improved patency and sealing, the flange configuration of FIG. 3D may be combined with either of the support structures illustrated in FIGS. 3A and 3B. In an alternative embodiment the flange 334 may sit inside the coronary sinus and form a seal with the vessel wall, preventing flow entering the right atrium.

FIGS. 3E and 3G illustrate venous collection devices 340, 350, each having a slightly different support structure which incorporates two consecutively inflatable regions. The first region is flange-like in shape and is configured to, when inflated, rest in abutment with a portion of the right atrium wall surrounding the coronary sinus ostium. When inflated, the second region sits inside the coronary sinus, contacting the vessel wall to form a seal and occlude flow between the sinus and the right atrium, whilst maintaining patency of the sinus while fluid is collected by the collection lumen. Collection lumen 308 extends through the balloon arrangement providing the added advantage of centralizing the tip 306 with respect to the sinus, further reducing the risk of invagination into the vessel wall and collapsing of the sinus.

To deploy such a device, a guide wire or catheter (not shown) placed inside the coronary sinus guides the inflatable support structure into position. The first region is inflated and a collection catheter extending therethrough is moved toward the coronary sinus ostium. Location of the ostium can be determined by deformation of the first region. When the first region is inflated with a fluid which includes a contrast solution, radiographic imaging may be used. When in position, the second region is inflated to occlude flow between the sinus and the right atrium, maintaining patency of the sinus and centralizing the catheter tip for collection of fluid. This may be achieved by inflating two separate balloons or two conjoined inflatable balloon regions.

The former requires incorporation of 3 lumens to enable (i) collection of fluid from the sinus; (ii) inflation of the first balloon; and (iii) inflation of the second balloon whereas the latter requires only 2 lumens.

In the embodiments illustrated in FIGS. 3E and 3G, the first and second inflatable regions are distinguished by a pressure-sensitive actuator which facilitates consecutive inflation of the first and second regions. When the first region has been inflated and a pre-determined pressure differential is established across the actuator, inflation of the second region occurs. The actuator may be in the form of a valve, membrane or conduit system. Advantageously, in the embodiments illustrated in FIGS. 3E and 3G, the first inflating region acts like an anchor, precisely locating the inflation site of the second inflating region.

Referring now to the particular embodiment illustrated in FIG. 3E, the first region is provided in the form of a first inflatable body 342 and the second region is provided in the form of a second inflatable body 344. The two regions are connected by a neck 346. In such an arrangement, the distance between the first and second inflatable bodies is pre-determined by the length of the neck, and this can be used to position the second inflatable body in the sinus accurately, prior to inflation. When the neck length is selected appropriately, occlusion of flow between the coronary sinus and the right atrium can be achieved whilst permitting collection of blood from substantially all of the tributary coronary veins feeding into the sinus, including the middle cardiac vein.

FIG. 3F is a cross sectional view illustrating how consecutive inflation of the first and second inflatable bodies may be achieved for the support structure illustrated in FIG. 3E. In such an arrangement, neck 346 connecting first and second inflatable bodies 342, 344 includes a secondary inflation conduit 348 which is much narrower than main inflation conduit 341. When the first inflatable body is inflated to a certain pressure, fluid is then forced to escape the first body through the secondary inflation conduit enabling second inflation body to inflate.

The support structure illustrated in FIG. 3G is in the form of a bell with the first and second inflatable bodies, 352, 354 distinguished by a membrane 356 inside the structure. The flange portion (base) of the bell which houses the first inflatable body 352 is configured to sit with wall 350a in abutment with a portion of the right atrium wall surrounding the coronary sinus ostium. The second inflatable body 354 is provided in the dome of the bell. When a certain pressure develops inside first inflatable body 352, membrane 356 fails enabling flow of fluid into and inflation of second inflatable body 354.

Alternatively, a valve or other suitable actuator may be used in place of membrane 356. Such valves may permit bidirectional control of flow between the first and second inflating bodies facilitating easy removal of the structure at the end of a procedure. In another embodiment, the dome of the bell may be folded upon itself onto the flange portion for percutaneous delivery to the coronary sinus. In such arrangement, a membrane or valve may not be necessary as adhesion between the folded layers may be sufficient to facilitate differential (consecutive) inflation of the two parts. The second inflating region may also include securing ribs, abrasions, spikes or the like, 343 to enhance stability of the support structure inside the sinus.

In dual balloon arrangement of the embodiments illustrated in FIGS. 3E and 3G, the first region has the capacity to provide a second level of occlusion, in addition the occlusion provided by the region of the balloon which is inflated inside the sinus. Guide wires and/or any other suitable ancillary devices may be used to deploy the inflatable support structure.

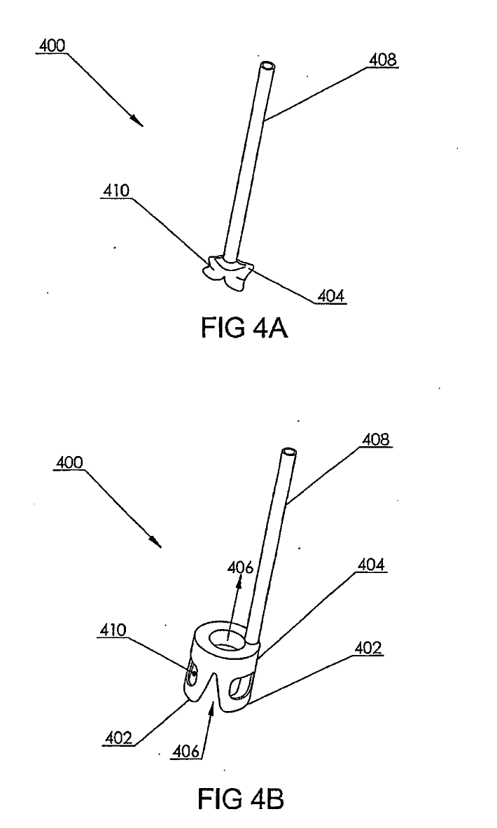

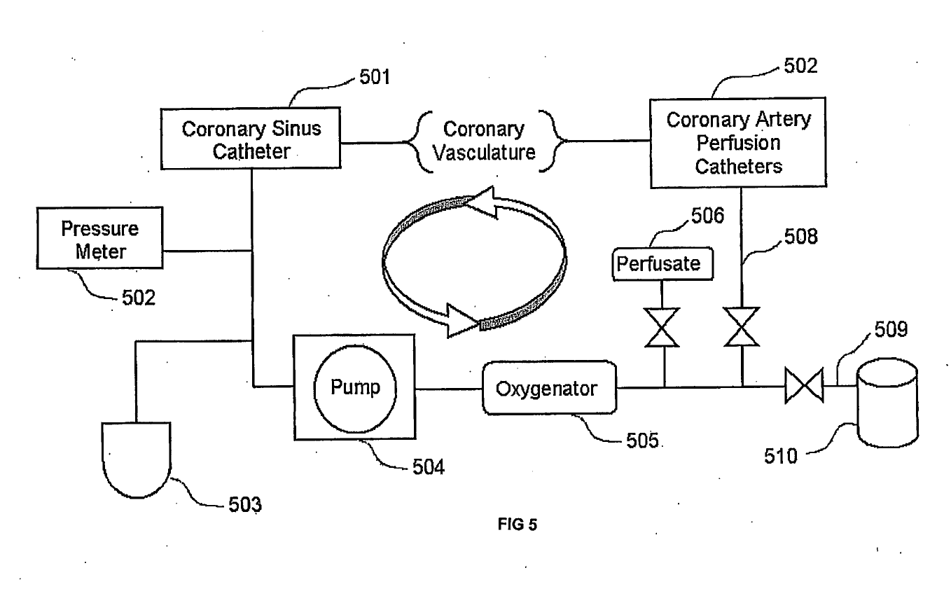

At the other end of the artificial flow path, a delivery device is positioned proximal the aortic valve to deliver blood (and agents) from the artificial flow path to the coronary arteries for circulation through the cardiac tissue. FIGS. 4A and 4B illustrate an example of a delivery device for delivering flow from the artificial flow path to the coronary arteries. The illustrated embodiment occludes flow between the aorta and the coronary arteries thereby substantially isolating the cardiac venous circulation from the systemic circulation. However, it is to be understood that the delivery device may deliver flow from the artificial flow path with or without occluding flow of fresh systemic blood from the aorta into the coronary arteries. In some embodiments where fluid from the artificial flow path is delivered to the coronary arteries at a relatively low flow rate, it may be desirable to permit extra blood flow from the aorta into the coronary arteries to supplement flow to the cardiac tissue. This results in dilution of any therapeutic agent introduced via the artificial flow path. However, such dilution can be compensated by replenishing supply of the agent in the artificial flow path.

In other embodiments, dilution of therapeutic agent may be undesirable so flow from the aorta into the coronary arteries should be prevented or at least minimized, thus resulting in the cardiac circulation being completely or substantially isolated from the systemic circulation. Accordingly, it may be desirable for the delivery device to occlude flow between the aorta and the coronary arteries. In a similar manner to the positioning of the venous collection device in the coronary sinus, the steps of positioning the delivery device and occluding flow between the aorta and the one or more coronary arteries may be performed in two separate steps or substantially simultaneously. Where these steps are performed substantially simultaneously, the delivery device may also be an occluding device. To achieve isolation of the artificial flow path supplying the coronary arteries from the systemic circulation, an occluding catheter may be used.

In one embodiment an occluding catheter for occluding flow between a main vessel and one or more branched vessels has a supply lumen and an inflatable body portion fed by the supply lumen. When inflated, the inflatable body portion occludes flow between the main vessel (aorta) and the one or more branched vessels (coronary arteries). The inflatable body portion has an opening which, when the body portion is inflated, creates one or more first flow channels between the supply lumen and one or more branched vessels (coronary arteries). When inflated, the inflatable body portion also forms a second flow channel which permits flow in the main vessel (aorta) across the inflatable body portion in isolation from the one or more first flow channels.

FIGS. 4A and 4B show a delivery and occluding device 400 according an embodiment of the present invention. An inflatable annulus 404 is shown in an un-inflated state in FIG. 4A and in an inflated state in FIG. 4B. Inflatable annulus 404 is fed with fluid from the artificial flow path by delivery lumen 408 thereby causing it to inflate. Delivery lumen 408 is in turn fed by the artificial flow path (not shown). Inflatable annulus 404 includes an opening 410 configured to remain substantially closed when the delivery device is deployed to the aortic valve region, and to open when the annulus is in position and inflated.

Alternatively, the opening may extend around a substantial portion of an outer wall of annulus 404 providing a flow path between the delivery lumen 408 and coronary arteries extending from the aortic sinus. In such an arrangement, the opening may therefore also supply accessory conal branches which exist in some patients ensuring more complete treatment of the cardiac tissue by any therapeutic agent which is introduced via the artificial flow path. The delivery device may provide two (as illustrated) or more openings 410 positioned around annulus 404 in such a way that each opening is used to establish a flow path to the left main (LM) and right (R) coronary arteries separately and to accessory branches if present via additional openings (not shown). When inflated, delivery device 400 also provides a systemic flow channel 406 through the center of the annulus 404. Systemic flow channel 406 enables the heart to generate and maintain cardiac output to the rest of the body substantially without delivering arterial blood to the coronary vasculature.

In use, the delivery device 400 is delivered percutaneously, in a deflated state (FIG. 4A), to the aorta and deployed just inside the cusp of the aortic valve. When in position, the annulus 404 is slowly inflated using supply from the artificial flow path, through delivery lumen 408. During delivery and deployment of the venous collection and delivery devices, it is important that their location and orientation is monitored. This is particularly important when deploying the delivery device, to ensure that openings 410 are positioned around the coronary ostia to enable flow between the artificial flow path and the coronary arteries. If the annulus is positioned in such a way that one or more of the coronary ostia is covered, inflation of the annulus is likely to occlude flow to the coronary arteries and cause serious damage to the cardiac tissue. Imaging techniques known in the art may be used, as well as guide wires, to aid in positioning the device.

It is desirable that the delivery device is sized appropriately to ensure a snug fit inside the aorta. A snug fit minimizes leakage or backflow from the delivery lumen 408 into the aorta and substantially prevents flow from the aorta into the coronary arteries. In some cases this fit may be problematic because it impedes closure of the aortic valve leaflets. To address this issue, it is preferable that inflatable annulus 404 is contoured to prevent obstruction of valve closure. In such an embodiment, the delivery device further includes lobes 402 which correspond to each of the lobes of the aortic valve. Inclusion of the lobes enables the delivery device to be positioned snuggly inside the aortic valve whilst permitting valve closure.

A delivery device of the kind illustrated in FIGS. 4A and 4B is suitable for delivering a blood (and therapeutic agent) solution from the artificial flow path to the coronary arteries without delivering the solution to the systemic circulation. The blood/therapeutic agent solution may then be collected after passing through the heart tissue using a venous collection device, as described previously. Collected solution can then be re-circulated through the heart with re-oxygenated collected blood. Such perfusion method is beneficial as it maximizes the efficiency of the therapeutic agent by eliminating break down in the liver and stomach, and also reduces or eliminates the toxicity issues associated with specific treatments reaching non-target tissue. Re-circulation further improves the uptake of therapeutic agents by increasing the exposure time to the target tissue, improving the effectiveness of treatment and reducing the cost of treatment by limiting uncontrolled loss of therapeutic agent to the systemic circulation. Recirculation of the polynucleotide can be performed for a period of time, where the minimum amount of time is equal to or about less than 1 minute, 1, 2, 3, 4, 5, 10, 15, 20, 25, 30, 45, 60, 90, 120, 150, or 180 minutes, and the maximum amount of time is equal to or about: 1, 2, 3, 4, 5, 10, 15, 20, 25, 30, 45, 60, 90, 120, 150, 180, or more than 180 minutes. Preferably, the amount of time is less than or equal to about 10 minutes.

As an alternative to the delivery device of FIGS. 4A and 4B, coronary artery occluding catheters may be used to supply the coronary arteries with the blood solution from the artificial flow path, and occlude flow from the aorta into the coronary arteries. Such occluding catheters may separately cannulate, for example, the left circumflex and left anterior descending arteries with for example, a 4 or 5 French catheter, over a wire. Alternatively, if flow from the aorta into the coronary arteries is not isolated, the coronary arteries may be catheterized and connected to the artificial flow path using any method deemed suitable by the skilled addressee. Catheterisation of the LM artery is considered particularly effective.

Venous blood in the circulating solution may become dangerously oxygen-depleted after supplying oxygen to the cardiac tissue. Therefore, it is desirable to include in the artificial flow path an oxygenation system, preferably of the kind normally incorporated into a cardiopulmonary bypass system or extracorporeal membrane oxygenation (ECMO) or equivalent. Using such a system, venous blood collected from the coronary sinus is oxygenated in the artificial flow path prior to it being re-circulated back into the heart. Therapeutic agent in the blood can also be replenished before re-circulation. An example of a re-circulation model is illustrated in FIG. 5 where therapeutic agent is indicated as “perfusate”.

Means for circulating the blood solution in the artificial flow path (and through the cardiac tissue) may be provided in a range of different forms as would be appreciated by the skilled addressee. Such means may comprise a roller pump incorporated into the apparatus to generate the required pressure head to circulate the blood. The relative pressure between the pump head and the coronary sinus is desired to be in the range −50 to −80 mmHg. However, a range of pressures is possible, with the minimum pressure being lower than −120, approximately: −120, −110, −100, −90, −80, −70, −60, −50, −40, −30, −20, or −10 mm Hg, and the maximum pressure being approximately −120, −110, −100, −90, −80, −70, −60, −50, −40, −30, −20, or −10, or greater than −10 mm Hg. To perfuse the polynucleotide solution into the coronary arteries, the solution should be drawn from the venous collection device at a suitable rate. It has been found that a rate of approximately 120 to 200 milliliters per minute may be suitable for most adults although flow rates as high as 250 milliliters per minute or as low as 50 milliliters per minute may be required. It is to be understood that this rate is not limiting to the invention (nor is the suggested pressure head), and may be adjusted according to the size, age and condition of the patient, and the nature of the apparatus and components used. A range of flow rates is possible, with minimal flow rates being less than 50, approximately: 50, 60, 70, 80, 90, 100, 110, 120, 130, 140, 150, 160, 170, 180, 190, 200, 210, 220, 230, 240, 250, 260, 270, 280, 290, or 300 ml/min, and maximum flow rates being approximately: 50, 60, 70, 80, 90, 100, 110, 120, 130, 140, 150, 160, 170, 180, 190, 200, 210, 220, 230, 240, 250, 260, 270, 280, 290, or 300, or more than 300 ml/min.

An auxiliary flow channel connected to a back-up reservoir may also be provided to supplement flow in the artificial flow path, if constant flow at the desired rate cannot be achieved naturally or if a dangerously high negative pressure is being generated at the pump. The back up reservoir may contain a volume of saline solution, blood or other inert fluid suitable for recirculation in the cardiac tissue. Alternatively, the auxiliary flow channel may draw blood from the right atrium to supplement the flow rate from in the artificial flow path. Typically the auxiliary flow path is driven using a roller pump.

Preferably, the apparatus includes means in the artificial flow path to monitor and adjust flow rates generated by the pump to compensate for the low flow from the coronary sinus into the collection lumen which may otherwise cause a) invagination of the collection lumen tip into the coronary sinus wall, or b) the coronary sinus to collapse or occlude, or c) the tip of the collection lumen to collapse or occlude. This also ensures adequate supply to the coronary arteries although this may be supplemented with aortic blood.

FIG. 5 shows a recirculation model for delivery of polynucleotide to a cardiac cell. A collection lumen 501 is located in the coronary sinus. A pressure meter 502 is located in the apparatus to monitor pressure in the venous flow path to assess the adequacy of venous drainage into the collection lumen. A pressure lower than, for example, −150, or −140 mmHg may be indicative of inadequate drainage and therefore inadequate flow. Similarly, a flow rate of less than about 80 ml/min may be indicative of an insufficient flow rate. This may be corrected by adding volume to the fluid in the circuit from the auxiliary flow path (e.g. a saline bag 503), or by adjusting the control at pump 504 to decrease the flow rate.

Blood collected from the coronary sinus is then oxygenated by oxygenator 505, ensuring air bubbles are removed to prevent transmission of air emboli into the perfusion circuit, and into the coronary circulation. Perfusate 506 is controllably introduced via a manifold or other suitable introducing apparatus at a rate that is suitable for achieving the desired therapeutic result. The perfusate introduction rate may be adjusted during perfusion, and preferably connectors are provided in the artificial flow path for sampling and analysis of fluid therein. Such analysis may include, for example, blood-gas analysis.

During recirculation, oxygenated perfusate and blood is recirculated into the coronary vasculature through open flow path 508 and one or more coronary artery perfusion catheters 507.

At completion of treatment, flow path 508 is closed (e.g. by clamping or valve closure) and flow path 509 is opened facilitating collection of remaining blood and perfusate in vessel 510. When normal flow of blood in the coronary vasculature resumes (when no more perfusate remains in the coronary sinus) the collection lumen is removed and the tubing and vessel 510 appropriately disposed of in biohazard waste.

Therapeutic agents and substances which may be added to the solution in the flow path may be selected from the group consisting of one or more of a virus, a pharmaceutical, a peptide, a hormone, a stem cell, a cytokine, an enzyme, a polynucleotide, blood and blood serum. Systemic exposure to therapeutic substances such as polynucleotides can result in the generation of a cellular or humoral immune response against the polynucleotide delivery vector backbone, resulting in elimination of the target transduced cell (i.e., cardiomyocyte overexpressing the phospholamban analog S16E). (Hernandez 1999, J. Virol. 73(10), 8549). Also, upon repeat administration, a previously generated immune response can block gene transfer upon subsequent administrations (Sanftner 2004, Mol. Ther. 9(3), 403; Moskalenko 2000, J. Virol. 74(4), 1761; Peden 2004, J. Virol. 78(12), 6344; Cottard 2004, J. Clin. Immunol. 24(2), 162).

Advantageously, the present invention enables treatment of the heart tissue by a therapeutic polynucleotide or the like, in the beating heart. This method of administration can use significantly less polynucleotide than previous cardiac transfection methods, while at the same time reducing or eliminating systemic exposure to the transfecting agent and thus reducing side effects and systemic immune response. Moreover, the effect can be long lasting and may result in transfection of a significant percentage of cardiac tissue. In fact, the achievable level of transfected cardiac tissue using the current invention is significantly higher than any method currently known in the art. Furthermore, the polynucleotide is substantially prevented from contact with non-cardiac cells. To the best of the applicant's knowledge, such treatment has not hitherto been achievable.

As used herein, “polynucleotide” has its ordinary and customary meaning in the art and includes any polymeric nucleic acid such as DNA or RNA molecules, as well as chemical derivatives known to those skilled in the art.

Examples of diseases intended to be treated using the present invention that are associated with the cardiovascular system include, but are not limited to, heart failure, ischemia, arrhythmia, myocardial infarction, congestive heart failure, transplant rejection, abnormal heart contractility, abnormal Ca²⁺ metabolism and congenital heart disease. Polynucleotides include not only those encoding a therapeutic protein, but also include sequences that can be used to decrease the expression of a targeted nucleic acid sequence using techniques known in the art (e.g., antisense, interfering, or small interfering nucleic acids). One example is a sequence which reduces or eliminates the expression of phospholamban. Polynucleotides can also be used to initiate or increase the expression of a targeted nucleic acid sequence or the production of a targeted protein within cells of the cardiovascular system. Targeted nucleic acids and proteins include, but are not limited to, nucleic acids and proteins normally found in the targeted tissue, derivatives of such naturally occurring nucleic acids or proteins, naturally occurring nucleic acids or proteins not normally found in the targeted tissue, or synthetic nucleic acids or proteins. One or more polynucleotides can be used in combination, administered simultaneously and/or sequentially, to increase and/or decrease one or more targeted nucleic acid sequences or proteins.

As used herein, “treat” or “treatment” of disease has its ordinary and customary meaning in the art and includes the cure, or less than complete cure of a disease, including the halting or slowing of the progression of a disease. The term “prevention” includes complete or incomplete prevention, or a delay of the onset of, a disease or the symptoms of a disease. The term “therapeutic effect” includes both treatment and prevention effects.

As used herein, “exogenous” nucleic acids or genes are those that do not occur in nature in the vector utilized for nucleic acid transfer; e.g., not naturally found in the viral vector, but the term is not intended to exclude nucleic acids encoding a protein or polypeptide that occurs naturally in the patient or host, e.g., SERCA.

As used herein, “cardiac cell” includes any cell of the heart that is involved in maintaining a structure or providing a function of the heart, such as a cardiac muscle cell, a cell of the cardiac vasculature, or a cell present in a cardiac valve. Cardiac cells include cardiomyocytes (having both normal and abnormal electrical properties), epithelial cells, endothelelial cells, fibroblasts, cells of the conducting tissue, cardiac pacemaking cells, and neurons.

As used herein, “substantially isolated” and its variants is a term that is not intended to require absolute isolation of the coronary venous, cardiac, systemic venous, or systemic circulation; rather, it is intended to mean that a majority, preferably the major part or even substantially all of the specified circulation is isolated.

As used herein, “preferentially exposed” has its ordinary meaning, and indicates that the targeted cell, tissue, or organ has a higher level of exposure to the administered substance than the non-targeted cell, tissue, or organ.

As used herein, “modulating” has its ordinary meaning, and encompasses both increasing and decreasing the expression or activity of a biological molecule.

As used herein, the term “minimally invasive” is intended to include any procedure that does not require open surgical access to the heart, or vessels closely associated with the heart. Such procedures include the use of endoscopic means to access the heart, and also catheter-based means relying on access via large veins, such as the femoral vein.

As used herein, the term “adeno-associated virus” or “AAV” encompasses all subtypes, serotypes and pseudotypes, as well as naturally occurring and recombinant forms. A variety of AAV serotypes and strains are known in the art and are publically available from sources, such as the ATCC, and academic or commercial sources. Alternatively, sequences from AAV serotypes and strains which are published and/or available from a variety of databases may be synthesized using known techniques.

As used herein, the term “serotype” refers to an AAV which is identified by and distinguished from other AAVs based on capsid protein reactivity with defined antisera. There are at least eight known serotypes of human AAV, including AAV-1 through AAV-8. For example, AAV-2 serotype is used to refer to an AAV which contains capsid proteins encoded from the cap gene of AAV-2 and a genome containing 5′ and 3′ inverted terminal repeat (ITR) sequences from the same AAV-2 serotype.

A “pseudotyped” AAV refers to an AAV that contains capsid proteins from one serotype and a viral genome including 5′ and 3′ inverted terminal repeats (ITRs) of a different or heterologous serotype. A pseudotyped rAAV would be expected to have cell surface binding properties of the capsid serotype and genetic properties consistent with the ITR serotype. A pseudotype rAAV may comprise AAV capsid proteins, including VP1, VP2, and VP3 capsid proteins, and ITRs from any serotype AAV, including any primate AAV serotype from AAV-1 through AAV-8, as long as the capsid protein is of a serotype heterologous to the serotype(s) of the ITRs. In a pseudotype rAAV, the 5′ and 3′ ITRs may be identical or heterologous. Pseudotyped rAAV are produced using standard techniques described in the art.

A “chimeric” rAAV vector encompasses an AAV vector comprising heterologous capsid proteins; that is, a rAAV vector may be chimeric with respect to its capsid proteins VP1, VP2 and VP3, such that VP1, VP2 and VP3 are not all of the same serotype AAV. A chimeric AAV as used herein encompasses AAV wherein the capsid proteins VP1, VP2 and VP3 differ in serotypes, including for example but not limited to capsid proteins from AAV-1 and AAV-2; are mixtures of other parvo virus capsid proteins or comprise other virus proteins or other proteins, such as for example, proteins that target delivery of the AAV to desired cells or tissues. A chimeric rAAV as used herein also encompasses a rAAV comprising chimeric 5′ and 3′ ITRs. The present invention encompasses chimeric rAAV vectors that comprise ITRs from different AAV serotypes, for example AAV1 and AAV2, or a chimeric rAAV may comprise synthetic sequences.

rAAV viral vectors may be produced by any of a number of methods known in the art including transient transfection strategies as described in U.S. Pat. Nos. 6,001,650 and 6,258,595, which are herein incorporated by reference. Typically, rAAV vector production requires three common elements; 1) a permissive host cell for replication which includes standard host cells known in the art including 293-A, 293-S (obtained from BioReliance), VERO, and HeLa cell lines which are applicable for the vector production systems described herein; 2) helper virus function which is supplied as a plasmid, pAd Helper 4.1 expressing the E2a, E4-orf6 and VA genes of adenovirus type 5 (Ad5) when utilized in transfection production systems; and 3) a transpackaging rep-cap construct. Transfection production may be performed as described in J Virology 2004 vol 78(22):12355-12365, incorporated by reference herein.

Methods of Polynucleotide Delivery

One aspect of the present invention contemplates transfer of therapeutic polynucleotide into a cell. Such transfer may employ viral or non-viral methods of gene transfer. This section provides a discussion of methods and compositions of gene or nucleic acid transfer, including transfer of antisense, interfering, and small interfering sequences.

In one form of the invention, the therapeutically significant polynucleotides are incorporated into a viral vector to mediate transfer to a cell. Additional expression constructs encoding other therapeutic agents as described herein may also be transferred via viral transduction using infectious viral particles, for example, by transformation with an adenovirus vector of the present invention. Alternatively, a retrovirus, bovine papilloma virus, an adeno-associated virus (AAV), a lentiviral vector, a vaccinia virus, a polyoma virus, or an infective virus that has been engineered to express may be used. Similarly, nonviral methods which include, but are not limited to, direct delivery of DNA such as by perfusion, naked DNA transfection, liposome mediated transfection, encapsulation, receptor-mediated endocytosis, and microprojectile bombardment may be employed. These techniques are well know to those of skill in the art, and the particulars thereof do not lie at the crux of the present invention and are thus need not be exhaustively detailed herein. However, in one preferred example, a viral vector is used for the transfection of cardiac cells to deliver a therapeutically significant polynucelotide to a cell. The virus may gain access to the interior of the cell by a specific means such receptor-mediated endocytosis, or by non-specific means such as pinocytosis.

A number of exemplary vectors will now be described. It will be appreciated that the following discussion is non-exhaustive

Adenoviral Vectors

A particular method for delivery of the expression constructs for gene therapy involves the use of an adenovirus expression vector. “Adenovirus expression vector” is meant to include those constructs containing adenovirus sequences sufficient to (a) support packaging of the construct and/or (b) to ultimately express a tissue and/or cell-specific construct that has been cloned therein.

In one form of the invention, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, Seminar in Virology, 3:237-252 (1992)). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and/or packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a preferred form of the method, recombinant adenovirus is generated from homologous recombination between shuttle vector and provirus vector. Due to the possible recombination between two proviral vectors, wild-type adenovirus may be generated from this process. Therefore, it is important to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B). Since the E3 region is dispensable from the adenovirus genome, the current adenovirus vectors, with the help of 293 cells, carry foreign DNA in either the E1, the D3 and/or both regions (Graham and Prevec, “Manipulation of adenovirus vectors,” Gene Transfer and Expression Protocols, Murray, E. J., ed., Humana, N. J., vol. 7, 109-128, (1991)). Recently, adenoviral vectors comprising deletions in the E4 region have been described (U.S. Pat. No. 5,670,488, incorporated herein by reference).

In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, and about 15% of the total length of the vector. More than 80% of the adenovirus viral genome remains in the vector backbone.

Helper cell lines may be derived from human cells such as embryonic kidney cells, muscle cells, hematopoietic cells or other embryonic mesenchymal and epithelial cells. Alternatively, the helper cells may be derived from the cells of other mammalian species that are permissive for human adenovirus. Such cells include, e.g., Vero cells or other monkey embryonic mesenchymal or epithelial cells.

Racher et al., Biotechnology Techniques, 9:169-174 (1995)) discloses improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, and/or at least conditionally defective, the nature of the adenovirus vector is not believed to be crucial to the successful practice of the present invention. The adenovirus may be of any of the 42 different known serotypes and/or subgroups A-F.

As stated above, the typical vector according to the present invention is replication defective and will not have an adenovirus El region. Thus, it was most convenient to introduce the transforming construct at the position from which the E1-coding sequences have been removed. However, the position of insertion of the construct within the adenovirus sequences is not believed critical to the invention. The polynucleotide encoding the gene of interest may also be inserted in lieu of the deleted E3 region in E3 replacement vectors as described by Karlsson et al. EMBO J. 5:2377-2385 (1986) or in the E4 region where a helper cell line and helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Adenovirus vectors have been used in eukaryotic gene expression and vaccine development. Recently, animal studies suggested that recombinant adenovirus could be used for gene therapy (see, e.g., Strafford-Perricaudet et al., Hum. Gene. Ther. 1:242-256 (1991); Rich et al., 1993). Studies in administering recombinant adenovirus to different tissues include muscle injection, peripheral intravenous injections and stereotactic inoculation into the brain. Recombinant adenovirus and adeno-associated virus (see below) can both infect and transduce non-dividing human primary cells.

Adeno-Associated Virus Vectors

Adeno-associated virus (AAV) is an attractive vector system for use in the cell transduction of the present invention as it has a high frequency of integration and it can infect non-dividing cells, thus making it useful for delivery of genes into mammalian cells, for example, in tissue culture and in vivo. AAV has a broad host range for infectivity. Details concerning the generation and use of rAAV vectors are described in U.S. Pat. No. 5,139,941 and/or U.S. Pat. No. 4,797,368, each incorporated herein by reference.

Studies demonstrating the use of AAV in gene delivery include Flofte et al., Proc. Natl. Acad. Sci. USA 90:10613-17 (1993) and Walsh et al., J. Clin. Invest. 94:1440-48 (1994). Recombinant AAV vectors have been used successfully for in vitro and in vivo transduction of marker genes and genes involved in human diseases (see for example, Walsh et al., J. Clin. Invest. 94:1440-48 (1994)).

AAV is a dependent parvovirus in that it requires coinfection with another virus (either adenovirus and/or a member of the herpes virus family) to undergo a productive infection in cultured cells. In the absence of coinfection with helper virus, the wild type AAV genome integrates through its ends into human chromosome 19 where it resides in a latent state as a provirus. rAAV, however, is not restricted to chromosome 19 for integration unless the AAV Rep protein is also expressed. When a cell carrying an AAV provirus is superinfected with a helper virus, the AAV genome is “rescued” from the chromosome and/or from a recombinant plasmid, and a normal productive infection is established.

Typically, recombinant AAV (rAAV) virus is made by cotransfecting a plasmid containing the gene of interest flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. rAAV virus stocks made in such fashion are contaminated with adenovirus which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used.

Retroviral Vectors

Retroviruses may be chosen as gene delivery vectors due to their ability to integrate their genes into the host genome, transferring a large amount of foreign genetic material, infecting a broad spectrum of species and cell types and of being packaged in special cell-lines.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and/or env genes but without the LTR and/or packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination.

Gene delivery using second generation retroviral vectors has been reported. Kasahara et al., Science 266:1373-1376 (1994), prepared an engineered variant of the Moloney murine leukemia virus, that normally infects only mouse cells, and modified an envelope protein so that the virus specifically bound to, and infected cells bearing the erythropoietin (EPO) receptor. This was achieved by inserting a portion of the GPO sequence into an envelope protein to create a chimeric protein with a new binding specificity.

Herpes Virus

Because herpes simplex virus (HSV) is neurotropic, it has generated considerable interest in treating nervous system disorders. Moreover, the ability of HSV to establish latent infections in non-dividing neuronal cells without integrating in to the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosing. For a review of HSV as a gene therapy vector, see (Glorioso et al., Annu. Rev. Microbiol. 49:675-710 (1995)).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotide reductase, a DNA polymerase, a ssDNA binding protein, a helicase/primase, a DNA dependent ATPase, dUTPase and others.

HSV genes form several groups whose expression is coordinately regulated and sequentially ordered in a cascade fashion. The expression of a genes, the first set of genes to be expressed after infection, is enhanced by the virion protein number 16, or α-transducing factor. The expression of β genes requires functional a gene products, most notably ICP4, which is encoded by the α4 gene. γ genes, a heterogeneous group of genes encoding largely virion structural proteins, require the onset of viral DNA synthesis for optimal expression.

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

Lentiviral Vectors

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-I, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see U.S. Pat. Nos. 6,013,516 and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc. and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Vaccinia Virus Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome. Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h.

Polyoma Viruses Vectors

The empty capsids of papovaviruses, such as the mouse polyoma virus, have received attention as possible vectors for gene transfer. The use of empty polyoma was first described when polyoma DNA and purified empty capsids were incubated in a cell-free system. The DNA of the new particle was protected from the action of pancreatic DNase. The reconstituted particles were used for transferring a transforming polyoma DNA fragment to rat FIII cells. The empty capsids and reconstituted particles consist of all three of the polyoma capsid antigens VP1, VP2 and VP3. U.S. Pat. No. 6,046,173 discloses the use of a pseudocapsid formed from papovavirus major capsid antigen and excluding minor capsid antigens, which pseudocapsid incorporates exogenous material for gene transfer.

Other Viral Vectors

Other viral vectors may be employed as expression constructs in the present invention, such as vectors derived from viruses such as sindbis virus or cytomegalovirus. They offer several attractive features for various mammalian cells (see e.g., Friedmann, Science 244:1275-1281 (1989); Horwich et al., J. Virol. 64:642-650 (1990)).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., J. Virol. 64:642-650 (1990)). This suggested that large portions of the genome could be replaced with foreign genetic material. Chang et al. introduced the chloramphenicol acetyltransferase (CAT) gene into duck hepatitis R virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., Hepatology 14:134A (1991)).

Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein or against a specific cell receptor were used. The antibodies were coupled via the biotin components by using streptavidin. Using antibodies against major histocompatibility complex class I and/or class II antigens, they demonstrated the infection of a variety of human cells that bore those surface antigens with an ecotropic virus in vitro.

Non-Viral Transfer

DNA constructs of the present invention are generally delivered to a cell, in certain situations, the nucleic acid to be transferred is non-infectious, and can be transferred using non-viral methods.

At least several non-viral methods for the transfer of expression constructs into cultured mammalian cells are contemplated by the present invention. Suitable methods for nucleic acid delivery for use with the current invention include methods as described herein or as would be known to one of ordinary skill in the art. Such methods include, but are not limited to, direct delivery of “naked” DNA plasmid via the vasculature (U.S. Pat. No. 6,867,196, incorporated herein by reference); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by agitation with silicon carbide fibers (Kaeppler et al., 1990; U.S. Pat. Nos. 5,302,523 and 5,464,765, each incorporated herein by reference) use of cationic lipids; naked DNA; or by microencapsulated DNA (U.S. Pat. Appl. No. 2005/0037085 incorporated herein by reference). Through the application of techniques such as these target cells or tissue can be stably or transiently transformed.

Once the construct has been delivered into the cell the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. The addition of DNA to cationic liposomes causes a topological transition from liposomes to optically birefringent liquid-crystalline condensed globules. These DNA-lipid complexes are potential non-viral vectors for use in gene therapy.

Liposome-mediated nucleic acid delivery and expression of foreign DNA in vitro has been very successful. Using the β-lactamase gene, investigators demonstrated the feasibility of liposome-mediated delivery and expression of foreign DNA in cultured chick embryo, HeLa, and hepatoma cells. Successful liposome-mediated gene transfer in rats after intravenous injection has also been accomplished. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-l. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993). Where liposomes are employed, other proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferring (Wagner et al., Proc. Natl. Acad. Sci. 87(9):3410-14 (1990)). A synthetic neoglycoprotein, which recognizes the same receptor as ASOR, has been used as a gene delivery vehicle and epidermal growth factor (EGF) has also been used to deliver genes to squamous carcinoma cells.

In other embodiments, the delivery vehicle may comprise a ligand and a liposome. For example, investigators have employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may he specifically delivered into a cell type such as cardiac cells, by any number of receptor-ligand systems with or without liposomes.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. It is envisioned that therapeutic DNA may also be transferred in a similar manner in vivo. Wolff et al. (U.S. Pat. No. 6,867,196) teach that efficient gene transfer into heart tissue can be obtained by injection of plasmid DNA solutions in a vein or artery of the heart. Wolff also teaches the administration of RNA, non-plasmid DNA, and viral vectors.

The vectors useful in the present invention have varying transfection efficiencies. As a result, the viral or non-viral vector transfects more than, equal to, or at least about 10%, 20%, 30%, 40%, 50%, 55%, 60%, 65%, 70%, 75%, 80%, 85%, 90%, 95%, 99%, 100% of the cells of the targeted vascular territory. More than one vector (viral or non-viral, or combination thereof) can be used simultaneously, or in sequence. This can be used to transfer more than one polynucleotide, and/or target more than one type of cell. Where multiple vectors or multiple agents are used, more than one transfection efficiency can result.

The therapeutic substances, including polynucleotides, that are useful in the present invention are utilized to treat cardiovascular disease. These substances include compounds known to treat any aspect of cardiovascular disease. The polynucleotide can target any of the known nucleic acids or proteins of the cardiovascular system. Of particular interest are the nucleic acids and proteins required for contraction of heat muscle, including the nucleic acids and proteins which regulate calcium concentrations in heart muscle.

Each muscle contraction requires the entry of Ca²⁺ into the muscle cytoplasm, while relaxation requires removal of the activating Ca²⁺, making Ca²⁺ regulation of muscle one of the most extensive activities in the human body. It is not surprising, therefore, that proteins involved in Ca²⁺ regulation, when mutated, can lead to a variety of skeletal and cardiac muscle diseases that are related to defects in Ca²⁺ regulation.

Sarcoplasmic reticulum calcium-ATPases pump calcium from the cytoplasm of mammalian cells into organellar structures such as the sarcoplasmic reticulum in muscle or the endoplasmic reticulum in non-muscle cells. Their threshold of activation by calcium is of the order of 100-200 nM, so that they set the resting level of cytoplasmic calcium. Abnormal calcium cycling, characteristic of experimental and human heart failure, is associated with impaired sarcoplasmic reticulum calcium uptake activity

SERCA2a, the cardiac/slow-twitch isoform binds to and is regulated by phospholamban (PLN), a 52 amino acid, homopentameric protein made up of three domains. During muscle contraction, phospholamban inhibits the Ca²⁺ pump. During muscle relaxation, it can be phosphorylated, which removes the inhibition and allows the Ca²⁺ to be pumped back into sarcoplasmic reticulum. The regulation is thought to be primarily from a physical interaction between the phospholamban monomer and the pump. However, the 52 residue peptide also associates into pentamers which have been shown to be selective for Ca²⁺ ions.

Impaired sarcoplasmic reticulum calcium uptake activity reflects decreases in the cAMP-pathway signaling and increases in type 1 phosphatase activity. The increased protein phosphatase 1 activity is partially due to dephosphorylation and inactivation of its inhibitor-1, promoting dephosphorylation of phospholamban and inhibition of the sarcoplasmic reticulum calcium-pump. Indeed, cardiac-specific expression of a constitutively active inhibitor-1 results in selective enhancement of phospholamban phosphorylation and augmented cardiac contractility at the cellular and intact animal levels. Notably, acute adenoviral gene delivery of the active inhibitor-1, completely restores function and partially reverses remodeling, including normalization of the hyperactivated p38, in the setting of pre-existing heart failure. (Pathak et al. Circ Res. 96(7):756-66 (2005)).

Ventricular arrhythmias can cause sudden cardiac death (SCD) in patients with normal hearts and in those with underlying disease such as heart failure. In animals with heart failure and in patients with inherited forms of exercise induced SCD, depletion of the channel-stabilizing protein calstabin2 (FKBP12.6) from the ryanodine receptor-calcium release channel (RyR2) complex causes an intracellular Ca²⁺ leak that can trigger fatal cardiac arrhythmias. Increased levels of calstabin2 stabilize the closed state of RyR2 and prevent the Ca²⁺ leak that triggers arrhythmias. Thus, enhancing the binding of calstabin2 to RyR2 is be a therapeutic strategy for common ventricular arrhythmias.

The nucleic acids and proteins of SERCA, phospholamban, and sarcolipin, as well as related nucleic acids and proteins which play a role in Ca²⁺, are preferred targets for polynucleotides of the present invention.

While it is contemplated that the present methods will be applicable to many animals, in preferred methods the animal is a large mammal such as a sheep or a human. Sheep are commonly used in drug and device efficacy evaluation studies of congestive heart failure. Sheep have hearts that are structurally similar to humans and lack the extensive collateral coronary circulation of other large animals. Rapid ventricular pacing is the most common animal model for the study of heart failure. In a preferred form of the method the subject is a human.

The invention will now be further described to the following non-limiting examples.

### EXAMPLE 1

### Recirculating Delivery of a Pseudophosphorylated Mutant of Phospholamban (S16E) Prevents the Progression of Heart Failure in a Large Animal Model

Using an embodiment of a cardiac isolation circuit described herein, the coronary venous circulation in a sheep was isolated from the systemic circulation. This isolated cardiac circuit provided a recirculating percutaneous system for the selective delivery of viral vectors to myocardium. Using this system, adenovirus (3.5×10¹²vp) encoding a pseudophosphorylated mutant of phospholamban was delivered to sheep as described below. The sequence of wild-type human phospholamban (PLN) is obtainable from Genbank under accession number NM_{—}002667, incorporated herein by reference. The pseudophosphorylated mutant used in this study (S16E) is a point mutation of phospholamban at amino acid 16 from S to E.

Animal Procedures

Day 0—Cardiac Pacemaker Implantation

Anesthesia and Site Preparation

The animal foreleg was clipped above the knee and a sterile area on the inside of the leg prepared using alcohol and a gauze swab. An 18 g Jelco catheter was placed into the foreleg vein and fixed in position with tape. Propofol (20 mL) was administered via the catheter. The animal was placed on the table and attached to the anesthetic machine. General anesthesia was administered with a continuous intravenous propofol (5 mg/kg/hr) and ketamine (15 mg/kg/hr) infusion. Animals was ventilated with 100% oxygen. LA tetracycline antibiotic (5 mL) and of finadyne analgesic (2 mL) was administered into the muscle of a hind leg. An ECG was obtained for monitoring purposes.

Pacemaker Implantation

After the proper depth of anesthesia is obtained, the neck was prepared by clipping the whole of one side of the neck and a surgical field prepared using alcohol and betadine. An incision (approximately 3 cm) was created over the jugular vein, using general sterile technique, and the vein exposed and surface cleared. A 2/0 silk purse string suture was placed in the wall of the vessel. A 54 cm pacing lead was removed from the pack under sterile conditions. A small incision was made in the vein within the purse string suture using a fine scalpel blade and the pacing lead immediately inserted into the incision and advanced until the lead collar was in the incision. The purse string was tightened slightly to stop any bleeding and the pacing lead advanced, using fluoroscopic guidance, until the lead was in the right ventricular apex. The lead was fixed in position and the purse string suture tightened and tied off.

A subcutaneous pocket was made under the skin of the flat of the neck above where the lead exits and the pacemaker lead connected to a Sigma pacemaker. The pacemaker was inserted into the pocket and sutured in place using the suture eyelet on the pacemaker. The skin incision was closed with 2/0 silk sutures, the wound sprayed with tetracycline spray and the neck bandaged with an elastic bandage.

Recovery

The implant site was inspected daily for any sign of wound breakdown or pacemaker migration. After 4 days the dressing was removed.

Days 5-33—Creating Heart Failure in Sheep by Rapid Pacing On Day 5, an echocardiographic examination was performed in the conscious animal and pacing commenced at 180 bpm. On Day 12, after one week of pacing at 180 bpm, pacing was stopped, and after 15 minutes an echocardiographic examination was performed to determine cardiac dimensions and function. Following the echocardiogram, pacing was restored to 180 bpm. An echocardiographic examination was performed weekly throughout the protocol.

Day 34—Documentation of Heart Failure and Vector Administration

After 28 days of pacing (Day 34) the animal was in heart failure and was ready to enter the treatment protocol. At this stage of heart failure there were minimal external symptoms except for a slightly raised respiratory rate. Animals were anesthetized as above. Sixteen animals with heart failure were assigned to two treatment groups.

Documentation and Measurement of Heart Failure

Animals were prepared as described herein. After the proper depth of anesthesia was obtained, echocardiogram and conductance catheter examinations were performed to confirm. heart failure.

Vector Administration

A 3 cm incision was made over the jugular vein and the vein exposed and the surface cleared. A 4/0 Ticron purse string suture was placed in the wall of the vessel and a 9 F sterile catheter introducer sheath placed in the vein, within the purse string suture. A 4/0 Ticron purse string suture was placed in the wall of the carotid artery and a 14 F sterile catheter introducer sheath placed in the artery, within the purse string suture. 10,000 IU heparin was administered to maintain an ACT of 300+ seconds. ACT measurement was done on the I-STAT equipment. Using an appropriate angiographic catheter, an angiogram was performed of the left and right coronary arteries to assess size of vessels and drainage area. If the right coronary artery (RCA) was small it was ignored. If the RCA is extensive a second introducer sheath was inserted in the carotid artery.

Under fluoroscopic guidance, one or two angiographic catheters were placed in the coronary ostia, as described herein, but not fully engaged. A 0.014 inch guide wire was placed within the vessel/vessels to anchor in place. The coronary sinus (CS) balloon catheter was placed in the CS and the catheter support structure was placed in the vessel at the distal tip.

The perfusion circuit was primed and prepared with normal saline, the circuit connected to the catheters and the pump started. Note that at this stage the coronary catheters are not engaged and CS catheter balloon was not inflated. The arterial end of the perfusion circuit (artificial flow path) was initially connected to a reservoir sidearm of a catheter introducer to enable priming of the circuit with blood to prevent the priming solution from being perfused directly into the coronary vasculature. Once the circuit was primed with blood, the coronary catheters were engaged and the balloon on the CS catheter inflated and perfused for 2 minutes. The pump rate was determined by the negative pressure within the circuit; a pressure of around −50 mmHg usually corresponded to a flow rate of 150-180 mL/min.

The AdS16EPLN was introduced into the circuit and perfused for 10 minutes. The ECG was monitored for abnormal rhythm and ECG profile during this period. The circuit was weaned-off by disengaging the coronary catheters. The blood in the reperfusion circuit containing the polynucleotide was collected by the CS catheter and drained into a receptacle for disposal. The balloon on the CS catheter and the catheters and the catheter introducer sheaths were removed and purse strings tightened. The skin incision was closed with 2/0 silk.

Day 48—Post-AAV2/1/SERCA2a vector Administration Pacing

Pacing was restarted at 180 bpm after the AAV2/1/SERCA2a vector administration and were continued for a maximum of 28 days.

Day 62—Terminal Sacrifice Procedures

Animals were prepared as described herein. After the proper depth of anesthesia was obtained animals were intubated. Cardiac function testing was performed by echocardiogram and conductance catheter, as described herein. Animals were euthanized with the intravenous administration of potassium chloride and organs weighed and histopathology samples collected.

Study Procedures

Echocardiogram

Pacing was stopped for at least 15 and not more than 30 minutes before the examination. The animal may be conscious (right side up, held on ground) or anaesthetized with right side up. Echocardiographic gel was placed on the skin of thorax and the examination performed. The minimum images include: long axis, short axis, short axis M mode at level of insertion of the papillary muscles and color Doppler of the mitral annulus. Ejection fraction, LV fractional shortening, LV freewall and septal dimensions, LV and LA long axis areas and MR assessment was computed and recorded. Other parameters may be measured as required.

Conductance Catheter Evaluation

Animals were prepared as described herein. Under fluoroscopic guidance an 8 F Fogarty balloon catheter was placed in the posterior vena cava and the conductance catheter (pre-calibrated) in the left ventricle. It was ensured that the tip of the catheter is not in contact with the LV wall by checking the ECG for extra stimuli. Acquiring on the system was commenced. The Fogarty catheter was inflated to occlude the vena cava. We continued to acquire data until the pressure trace dropped by one-third (i.e., for approximately 60 seconds). Data were analyzed to obtain LVEDP, LV ±dP/dt, LV End Systolic Pressure Volume Relationship (Ees).

Adenovirus (3.5×10¹²vp) encoding a pseudophosphorylated mutant of phospholamban (AdS16EPLN, n=9) or adenovirus encoding a control gene for β-Galactosidase (LacZ; AdLacZ (n=6, 4.7×10¹²vp) was delivered to sheep with pacing induced heart failure (described above and Aust N Z J Med. 29(3):395-402 (1999); and J Card Fail. 8(2):108-15 (2002)). Despite 2 weeks further pacing, treatment with AdS16E PLN significantly improved contractile function despite ongoing pacing stress and prevented ventricular remodeling in contrast to AdLacZ animals, as shown in the Table below.

| Parameter (% change vs | AdS16E | |
|---|---|---|
| baseline) | PLN | AdLacZ |
| LV End Diastolic Area | −14** | +13** |
| LV Ejection Fraction | +87*** | −23* |
| LV End Diastolic Pressure | −24* | +5 |
| dP/dt | +31* | −5 |

p < 0.05,
**p < 0.01,
***p < 0.001

### EXAMPLE 2

### Delivery of SERCA2a to Cardiac Cell

Using a the same technique as in Example 1 for isolating the coronary venous circulation and delivering viral vector to sheep myocardium, adenovirus was delivered (3.5×10¹²vp) encoding the sarcoplasmic reticulum ATPase 2a (AdSERCA2a (n=2)) or adenovirus encoding a control gene for β-Galactosidase (LacZ;AdLacZ (n=6, 4.7×10¹²vp) to sheep with pacing induced heart failure. The sequence of human SERCA2a can be obtained at Genbank accession numbers NM_{—}170665 and NM_{—}001681, incorporated herein by reference. Despite 2 weeks further pacing, treatment with AdSERCA2a significantly improved contractile function despite ongoing pacing stress and prevented ventricular remodeling in contrast to AdLacZ animals, as shown in FIG. 5.

### EXAMPLE 3

### Delivery of β-Galactosidase to a Cardiac Cell

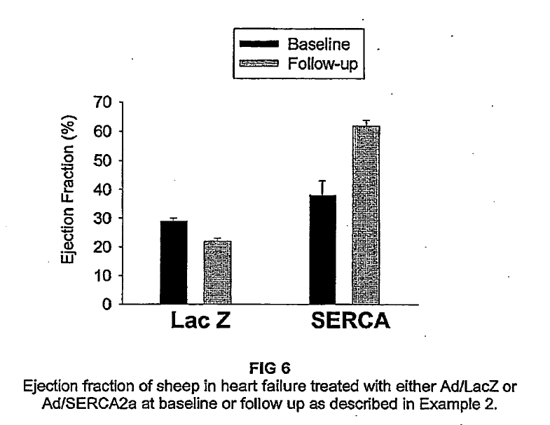

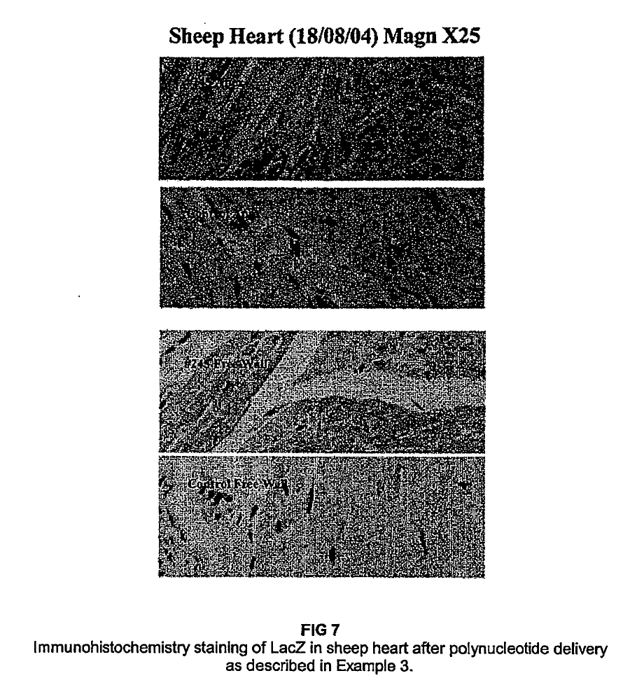

Using the same technique as in Example 1 for isolating the coronary venous circulation and delivering viral vector to sheep myocardium described in Examples 1 and 2, we delivered adeno-associated virus (2×10¹²vp) encoding the gene for β-Galactosidase (LacZ ) [AAV2/LacZ]. Immunohistochemical staining for β-Galactosidase in heart indicated high efficiency transduction of the majority of cardiomyocytes in treated vs. control, non-treated animals (see FIG. 6). The Ad/LacZ was made as described in Hoshijima, M., et al. Nat. Med. 8:864-871 (2002).

### EXAMPLE 4

### Investigation of Adverse Effects of Polynucleotide Introduction

Using the techniques described in Examples 1-3 we have demonstrated the safe, reproducible, delivery of adeno-LacZ to the myocardium of both healthy normal sheep (n=15) and sheep with moderate to severe pacing induced heart failure (n=7). During 10-15 minutes recirculation, there was no evidence of compromise in myocardial function (as assessed by echocardiography and electrocardiographic monitoring). Similarly, there was no evidence of lactic acid accumulation in the recirculating blood (baseline vs 15 mins: 2.4 mmol/L vs 2.5 mmol/L). A homogeneous pattem of LacZ delivery was apparent by immunohistochemistry, 2 weeks after gene transfer and no evidence of tissue damage was evident on H+E staining. Of additional importance this ‘closed-loop’ recirculation system results in minimal systemic leakage (brain, kidney, liver and lung) of viral vector.

### EXAMPLE 5

### Selective Delivery of SERCA2a to Cardiac Cells

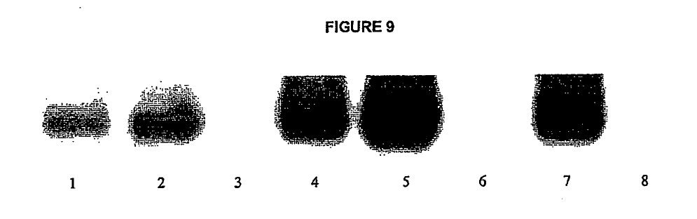

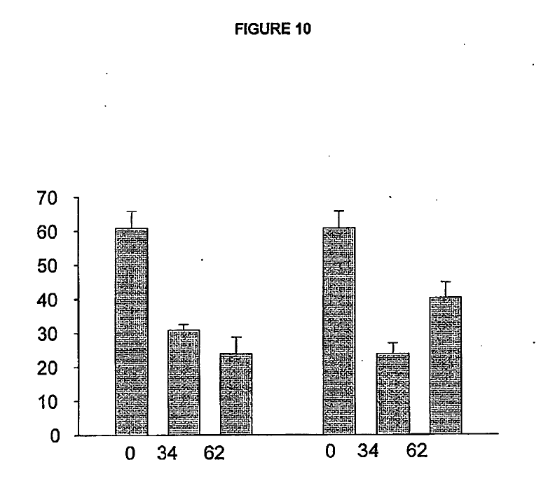

Using the same technique as in Example 1 for isolating the coronary venous circulation and delivering viral vector to sheep myocardium, adenovirus was delivered (3.5×10¹²vp) encoding the sarcoplasmic reticulum ATPase 2a (AdSERCA2a) or adenovirus encoding a control gene for β-Galactosidase (LacZ;AdLacZ (4.7×10¹²vp) to sheep with pacing induced heart failure. The sequence of human SERCA2a can be obtained at Genbank accession numbers NM_{—}170665 and NM_{—}001681. At the conclusion of a further 2 weeks pacing the animals were terminated. Samples of the left ventricular free wall and liver were obtained and rapidly frozen in liquid nitrogen. Samples were subsequently homogenized in a lysis buffer appropriate for protein analysis. Proteins were resolved by electrophoresis on a 10% SDS-PAGE gel. The resolved proteins were then transferred via Western transfer to a nitrocellulose membrane and probed for SERCA2a expression by immunoblolting using a commercially available antibody (Santa Cruz). Densitometric analysis of signals detected by autoradiography was then performed. This analysis indicated an increase in the expression of SERCA2a in the AdSECA2a animal by 1.9 fold as shown in FIG. 9. The Western blot also indicates that expression of SERCA2a was not apparent in the liver of the animal receiving AdSERCA2a.

### EXAMPLE 6

### Four Week, Single Dose, Safety and Efficacy Study of AAV2/1/SERCA2a using a Percutaneous Delivery Method in an Ovine Pacing Model of Heart Failure

The purpose of this study was to evaluate the efficacy (as determined by LVEF) and safety of AAV2/1/SERCA2a when delivered as a single administration via closed-circuit infusion between the coronary artery circulation and the coronary sinus in an ovine model of heart failure created by rapid right ventricular pacing. The study will also be used to assess the reversibility, persistence, or delayed occurrence of any effects after an additional 28 days of rapid pacing.

The study design included two treatment groups: one active treatment (AAV2/1/SERCA2a; 5×10¹²drp) percutaneous delivery system will be compared to a sham (no device, no AAV2/1/SERCA2a vector) control group. A total of 16 female sheep with heart failure (defined as having an ejection fraction of 25%) were used.

Polynucleotide delivery was achieved by closed circuit reperfusion between the coronary artery circulation and the coronary sinus. Route of administration was by intracoronary percutaneous delivery with recirculation

Study duration was 62 days total, with pacemaker implantation on Day 0, with pacing begun on Day 5, AAV2/1/SERCA2a was administered 28 days later, on Day 34, and animals sacrificed, on Day 62.

AAV2/1/SERCA2a Viral Vector

Information on synthesis methods, composition, or other characteristics that define the AAV2/1/SERCA2a vector is on file with the manufacturer, Targeted Genetics (Seattle, Wash.). The methods used to prepare the AAV serotype 1 containing ITRs from AAV serotype 2, huSERCA2A expression plasmids, and the encapsidated rAAV viral vector production and purification methods, are described briefly below.

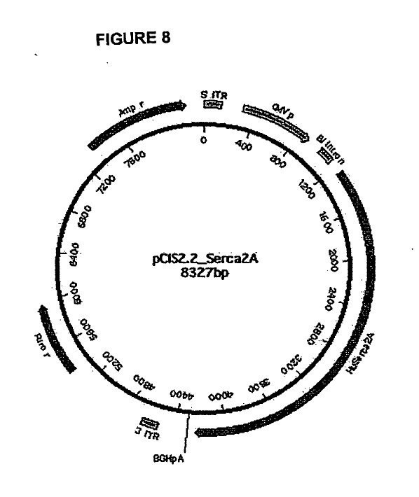

Generation of the of AAV ITR serotype 1 huSERCA2A expression plasmids The pcDNA3.1/HuSERCA2a plasmid was digestested with Nhel and Xhol restriction enzymes and a 2.3 Kb fragment containing the 5′ portion of the SERCA cDNA was isolated. The pCis2.2 plasmid containing the AAV2 5′ and 3′ITR sequences was digested with NheI and XhoI restriction enzymes to linearize plasmid. The 2.3 kb fragment containing the 5′ portion of the SERCA2a cDNA was ligated into NheI/XhoI digested pCis2.2. The 3′ UTR from the SERCA 2a plasmid was generated by PCR and flanked by XhoI restriction sites to yield a 1.1 kb 3′ SERCA2a fragment. The 1.1 kb 3′ SERCA2a fragment was ligated into XhoI digested pCis2.2/5′ SERCA2a to yield the final vector plasmid, pCis2.2 SERCA2a (see FIG. 8).

Encapsidated rAAV: Viral Vector Production and Purification Methods

Briefly, 293 cells were grown by seeding a 10 stack cell factory in DMEM media with 4 mM L-glutamine and 10% FBS. The flasks were incubated for 96 hours at. After 96 hours of incubation at 37° C. with 5% CO2, the cells were transfected using calcium phosphate. The cell factories were typically 80-90% confluent on the day of transfection. A transfection mixture containing the pBS-HSP-R2ClAAV Helper plasmid encoding the AAV I capsid and AAV2 rep gene, the Ad Helper 4.1 plasmid and of the pCic2.2_SERCA2a plasmid were added to a solution of 300 mM Calcium Chloride. The plasmid containing calcium chloride was slowly poured into 2× HBS buffer and allowed to mix for 30 seconds. The precipitate was immediately added to each cell factory. The flasks were placed at 37° C. with 5% CO2 for 6 to 8 hours. After 6-8 hours, the cell factory was removed from the incubator and the media removed from each of the flasks and replaced with fresh DMEM media with 4 mM L-glutamine. The cell factory was incubated for 72 hours at 37° C. with 5% CO₂.

After 72 hours, the cell factory was removed from the incubator and tapped to release the cells. The contents of the cell factory was pooled into roller bottles. 100 mM MgCl₂ and 10% DOC were added to achieve a final concentration of 1.8 mM and 0.5%, respectively. The roller bottles were placed in a 37° C. waterbath for 10-20 minutes. Benzonase was added to each roller bottle to achieve a final concentration of 10 units per mL. The roller bottles were placed in a 37° C. waterbath for 60 minutes, inverting every 15 minutes. Polysorbate 20 (Tween) was added to each roller bottle to achieve a final concentration of 1%. The roller bottles were placed in a 37° C. waterbath for 60 minutes, inverting every 15 minutes. 5M NaCl was added to each roller bottle to achieve a final salt concentration of 1M.

The lysed material was filtered through two filters, Polygard CR Optivap XL 10 filter (0.3 μm nominal) and Milligard Opticap Capsule (0.5 μm nominal). Once filtered, the material was concentrated using tangential flow filtration (TFF). Three TFF membranes were used with a 100 Kda nominal molecular weight cut off (NMWCO). The filtered material was concentrated to a volume of 500 mL. Once concentrated, the material was diafiltered with 10 diavolumes. The diafiltered material was then filtered through a Suporcap 50 liter (0.45 μm) and placed at −70° C. until purification. Purification of the psuedotyped vector was performed as described in J Virology 2004 vol 78(22):12355-12365.

The AAV2/1/SERCA2a vector was stored frozen at −70° C. or below until use. The vector was supplied as a 2.7×10¹² drp/mL stock solution. The morning of use, the stock solution of the AAV2/1/SERCA2a vector was thawed at room temperature.

After gentle mixing, stock solution was diluted by aseptically transferring 1.85 mL of AAV2/1/SERCA2a vector to a sterile polypropylene tube, and diluted with sterile human grade saline for IV use to a total volume of 10 mL (5×10¹¹ drp/mL). This intermediate solution was kept on ice until the animal was ready for dosing.

Just prior to dosing the animal, 10 mL of the solution from above was diluted with 10 mL whole blood from the animal, to a concentration of 2.5×10¹¹ drp/mL. (Each animal was dosed with 20 mL. of this diluted rAAV vector solution for a total dose of 5×10¹² drp per animal.)

Test Animals and Husbandry.

Sixteen female sheep (Merino×Border Leicester) were sourced from a commercial breeder. At initiation of treatment animals were 2-3 years of age and weighed from 50 to 70 kg.

Animals were individually penned in a specialized sheep facility and fed mixed lucerne and oaten chaff, 4 kg once daily in the morning. Water was provided ad libitum. Temperatures ranged between 20-30° C. with seasonal diurnal light cycles. Animals were acclimated for at least 14 days prior to start of the study.

Protocol

Day-14: Study Entry

Animals entered into the study were shorn, feet clipped, drenched, and ear tags attached.

Day 0: Cardiac Pacemaker Implantation

Animals were weighed and randomized to treatment with rAAV2/1/SERCA2a or control.

Sixteen animals were assigned by odd and even numbers to two treatment groups, Group #1 Control (no percutaneous procedure, no AAV2/1/SERCA2a vector,) Group #2 Percutaneous Procedure+AAV2/l/SERCA2a vector. The control group for this study was a sham surgery control. Animals in the sham surgery control group underwent all the procedures except for the percutaneous device intervention.

Anesthesia and Site Preparation

The animal foreleg was clipped above the knee and a sterile area on the inside of the leg prepared using alcohol and a gauze swab. An 18 g Jelco catheter was placed into the foreleg vein and fixed in position with tape. Propofol (20 mL) was administered via the catheter. The animal was placed on the table and attached to the anesthetic machine. General anesthesia was administered with a continuous intravenous propofol (5 mg/kg/br) and ketamine (15 mg/kg/br) infusion. Animals were ventilated with 100% oxygen. Vital signs (Heart rate, respiratory rate, and 0₂ pulse oximeter) were monitored for the duration of the procedure. LA tetracycline antibiotic (5 mL) and finadyne analgesic (2 mL) was administered into the muscle of a hind leg.

An ECO was obtained for monitoring purposes and an echocardiographic examination performed under anesthesia, as described herein.

Pacemaker Implantation

After the proper depth of anesthesia was obtained, the neck was prepared by clipping the whole of one side of the neck and a surgical field prepared using alcohol and betadine.

An incision (approximately 3 cm) was created over the jugular vein, using general sterile technique, and the vein exposed and surface cleared. A 2/0 silk purse string suture was placed in the wall of the vessel. A 54 cm pacing lead was removed from the pack under sterile conditions. A small incision was made in the vein within the purse string suture using a fine scalpel blade and the pacing lead immediately inserted into the incision and advanced until the lead collar was in the incision. The purse string was tightened slightly to stop any bleeding and the pacing lead advanced, using fluoroscopic guidance, until the lead was in the right ventricular apex. The lead was then be fixed in position and the purse string suture tightened and tied off.

A subcutaneous pocket was made under the skin of the flat of the neck above where the lead exits and the pacemaker lead connected to a Sigma pacemaker. The pacemaker was inserted into the pocket and sutured in place using the suture eyelet on the pacemaker. The skin incision was then closed with 2/0 silk sutures, the wound sprayed with tetracycline spray and the neck bandaged with an elastic bandage.

Recovery

The implant site was inspected daily for any sign of wound breakdown or pacemaker migration. After 4 days the dressing was removed.

Day 5: Rapid Pacing Commenced and Blood Samples

On Day 5 pacing commenced at 180 bpm and blood samples were collected from the jugular vein for baseline measurements of the following parameters: hematology/coagulation, clinical chemistry, cardiac enzymes, and inflammation markers.

Days 12-26: Check Pacing

Pacing was checked Day 12, after one week of pacing at 180 bpm, and again on Days 19, & 26.

Day 19: Check Pacing and Echocardiographic Exam

Pacing was checked and an echocardiographic examination performed in the conscious animal as described herein.

Day 34: Documentation of Heart Failure and Vector Administration

After 28 days of pacing (Day 34) the animal was typically in heart failure (as confirmed by documenting an ejection fraction of 25%). At this stage of heart failure there were typically minimal external symptoms except for a slightly raised respiratory rate. Pacing was stopped for the procedure and re-started on Day 35.

Pre-AAV2/1/SERCA2a Vector Administration Blood Collection

Blood samples were collected for hematology/coagulation, clinical chemistry, cardiac enzymes, and inflammation markers.

Documentation and Measurement of Heart Failure

Animals were weighed and prepared as described herein. After the proper depth of anesthesia was obtained, echocardiogram and conductance catheter examinations were performed to confirm heart failure (defined as ejection fraction 25%), as described herein.

Vector Administration

Animals randomized to receive AAV2/l/SERCA2a vector underwent the percutaneous procedure and received the vector. A 3 cm incision was made over the jugular vein and the vein exposed and the surface cleared. A 4/0 Ticron purse string suture was placed in the wall of the vessel and a 9 F sterile catheter introducer sheath placed in the vein, within the purse string suture.

10,000 IU heparin was administered to maintain an ACT of 300+ seconds. ACT measurement was done on the I-STAT equipment.

Using an appropriate angiographic catheter, an angiogram was performed of the left and right coronary arteries to assess size of vessels and drainage area. If the right coronary artery (RCA) was found to be small (<5 Fr), it was ignored. If the RCA is extensive a second introducer sheath was inserted in the carotid artery.

Under fluoroscopic guidance, one or two angiographic catheters were placed in the coronary ostia, as described herein, but not fully engaged. A 0.014 inch guide wire was placed within the vessel/vessels to anchor in place.

The coronary sinus (CS) balloon catheter was placed in the CS and the support structure placed in the vessel at the distal tip.

The perfusion circuit was primed and prepared with saline solution, the circuit connected to the catheters and the pump started. Note that at this stage the coronary catheters were not engaged and CS catheter balloon was not inflated. Once the circuit was primed with blood, the coronary catheters were engaged and the balloon on the CS catheter inflated and perfused for 2 minutes. The pump rate was optimised by monitoring the negative pressure within the circuit. Most typically the flowrates captured from the coronary sinus were between 100-130 ml/minute using this method.

The AAV2/1/SERCA2a vector was introduced into the circuit and perfused for 10 minutes. The ECG was monitored for abnormal rhythm and ECG profile during this period.

The circuit was emptied (approximately 120 mL) into a disposable bag and the animal weaned-off by disengaging the coronary catheters and deflating the balloon on the CS catheter. The catheters and the catheter introducer sheaths were then removed and purse strings tightened. The skin incision was closed with 2/0 silk.

The blood and the reperfusion circuit containing the AAV2/1/SERCA2a vector was disposed of in an appropriate biohazard container.

Day 35: Post-Treatment Pacing

Pacing was restarted on Day 35 at 180 bpm, after the AAV2/1/SERCA2a vector administration, and continued for a maximum of 28 days.

Day 37: Post-treatment Blood Collection

Blood samples were collected for hematology/coagulation, clinical chemistry, cardiac enzymes, and inflammation markers.

Days 41-55: Check Pacing

Pacing was checked on Days 41, 48, & 55, as described herein.

Day 49: Check Pacing and Echocardiographic Exam

Pacing was checked and an echocardiographic examination performed in the conscious animal, as described herein.

Day 62: Terminal Sacrifice Procedures

Animals were weighed and prepared as described herein.

Blood samples were collected for hematology/coagulation, clinical chemistry, cardiac enzymes, and inflammation markers.

Cardiac function testing was performed by echocardiogram and conductance catheter, as described herein.

Animals will then be euthanized with the intravenous administration of 30 mg/kg potassium chloride and histopathology samples collected.

Study Procedures

Echocardiogram

Pacing was stopped for at least 15 and not more than 30 minutes before the examination. The animal was typically conscious (right side up, held on ground) or anaesthetized with right side up. Echocardiographic gel was placed on the skin of thorax in the axillary region and the examination performed. The minimum images include: long axis, short axis, short axis M mode at level of insertion of the papillary muscles and color Doppler of the mitral annulus. Ejection fraction, LV fractional shortening, LV freewall and septal dimensions, LV and LA long axis areas and MR assessment will be computed and recorded. Other parameters were measured as required.

Following the echocardiogram, pacing was restored to 180 bpm.

Conductance Catheter Evaluation

Animals were prepared as described herein. Under fluoroscopic guidance an 8 F Fogarty balloon catheter was placed in the posterior vena cava and the conductance catheter (pre-calibrated) in the left ventricle. It was ensured that the tip of the catheter was not in contact with the LV wall by checking the ECG for extra stimuli.

Acquiring on the system was then commenced. The Fogarty catheter was inflated to occlude the vena cava. Data was continued to be acquired until the pressure trace dropped by one-third (i.e., for approximately 60 seconds). Data was analyzed to obtain LVEDP, LV±dP/dt, LV End Systolic Pressure Volume Relationship (Ees).

Cardiac Function Testing was performed using the methods of echocardiogram and conductance catheter.

Sacrifice and Tissue Collection

Animals will be sacrificed under anesthesia with an intravenous administration of 30 mg/kg potassium chloride. The following samples will be collected and put in 10% neutral formalin for histopathological examination: aorta, brain (6 slices including brain stem), heart (bread slice into 5 slices, RV, septum and LV freewall from each), kidney (2), liver, lung with bronchi, lymph nodes (mesenteric and mediastinal), ovary (2), skin, spleen.

Clinical Pathology

Frequency and Description of Tests

Four (4) tubes of blood were collected on Day 5, Day 34, prior to AAV2/1/SERCA2a vector administration, Day 37, Day 49 and on Day 62 prior to terminal sacrifice for: (1) hematology/coagulation, (2) clinical chemistry, (3) cardiac enzymes, (4) inflammation markers. In addition duplicate 500 serum samples were taken at each blood sampling interval from each animal and stored at −20“C. for possible additional analysis at a later date.

Hematology: leukocyte count, erythrocyte count, hemoglobin, hematocrit, MCH (mean corpuscular hemoglobin), MCV (mean corpuscular volume), MCHC (mean corpuscular hemoglobin concentration), platelet count, differential count, reticulocyte count

Coagulation: PT (Prothrombin Time), APTT (Activated Partial Thromboplastin Time)

Clinical Chemistry: sodium, potassium, chloride, total protein, albumin, calcium, phosphorus, total bilirubin, urea nitrogen, creatinine, glucose, total cholesterol, ALT, AST, alkaline phosphatase, triglycerides, globulin, A/G ratio (calculated)

Cardiac Enzymes: CPK, troponin I

Inflammation Markers: C-Reactive Protein (CRP)

Histopathology

The following tissues will be collected at the scheduled necropsy on Day 62 and put in 10% neutral formalin. Cardiovascular (aorta, heart) urogenital (kidneys, ovaries, urinary, bladder, uterus, cervix, vagina); endocrine (adrenals, pituitary, thyroid/parathyroid); skin/musculoskeletal (skin, mammary gland, skeletal muscle (thigh), femur with articular surface); Nervous/Special Sense (eye with optic nerve, sciatic nerve, brain, cervical spinal cord, midthoracic spinal cord, lumbar spinal cord, lacrimal glands); digestive (salivary gland(s), tongue, esophagus, stomach, small intestine, duodenum, jejunum, ileum, large intestine, cecum, colon, rectum, pancreas, liver, gallbladder); Respiratory (trachea, larynx, lung with mainstem bronchus); lymphoid/hematopoietic (sternum with bone marrow), thymus, spleen, lymph nodes (mandibular and mesenteric)); Other (unique animal identifier (not for evaluation), gross findings, bone marrow smears (from rib or sternum)).

Tissue samples and bone marrow smears (stored, packaged, and shipped separately) will be embedded in paraffin, sectioned, stained with hematoxylin and eosin, and examined microscopically: aorta, brain (6 slices including brain stem), heart (bread slice into 5 slices, RV, septum and LV freewall from each), kidney (2), liver, lung with bronchi, lymph nodes (mesenteric and mediastinal), ovary (2), skin, spleen.

A schedule of study procedures is provided in the table below.

| | Study | Pacemaker | | | | Test | | | Terminal |
|---|---|---|---|---|---|---|---|---|---|
| | Entry | Implant | | | | Article | | | Sacrifice |
| Evaluation | Day −14 | Day 0 | Day 5 | Weekly | Day 19 | Day 34¹ | Day 37 | Day 49 | Day 62 |
| Selection of study | X | | | | | | | | |
| animals² | | | | | | | | | |
| Clinical | X | X | X | | X | X | | X | X |
| observation & feed | | | | | | | | | |
| intake³ | | | | | | | | | |
| Randomization | | X | | | | | | | |
| Anesthesia and | | X | | | | X | | | X |
| site preparation | | | | | | | | | |
| Body weight⁴ | | X | | | | X | | | X |
| LA tetracycline & | | X | | | | X | | | |
| finadyne | | | | | | | | | |
| ECG monitoring | | X | | | | X | | | X |
| Vital signs⁵ | | X | | | | X | | | X |
| Blood samples⁶ | | | X | | | X | X | X | X |
| Anticoagulation to | | | | | | X | | | |
| ACT 300+ sec | | | | | | | | | |
| Pacemaker | | X | | | | | | | |
| implantation | | | | | | | | | |
| Start pacing | | | X | | | | | | |
| Echo under | | X | | | | X | | | X |
| anesthesia | | | | | | | | | |
| Check pacing⁷ | | | | X | | | | | |
| Conscious echo | | | | | X | | | X | |
| Conductance | | | | | | X | | | X |
| catheter | | | | | | | | | |
| examination | | | | | | | | | |
| Vector | | | | | | X | | | |
| administration | | | | | | | | | |
| Terminal sacrifice | | | | | | | | | X |
| Tissue collection | | | | | | | | | X |
| Tissue samples for | | | | | | | | | X |
| histopathology | | | | | | | | | |

¹Pacing was stopped on Day 34, for vector administration, and re-started on Day 35.
²Animals were acclimated for at least 34 days prior to the start of the study. At Day −14 animals were shorn, feet clipped, drenched and ear tags attached.
³Animals were observed daily for failure to eat or drink, weakness or any other form of distress.
⁴Animals were weighed on Day 0, Day 34 and prior to terminal sacrifice on Day 62.
⁵Heart rate, respiratory rate, and 0₂ pulse oximeter.
⁶Hematology/coagulation, clinical chemistry, cardiac enzymes, and inflammation markers. In addition duplicate 500 μL serum samples were taken at each blood sampling interval from each animal and stored at −20° C. for possible additional analysis at a later date.
⁷Pacing was checked weekly on Days 12, 19, 26, 41, 49, & 55

Statistical Evaluation

The sample size of 8 animals per treatment group was expected to be sufficient to assess efficacy and safety of AAV2/1 /SERCA2a.

Animals were randomized to either active treatment (AAV2/l/SERCA2a) percutaneous delivery system or sham (no device, no AAV2/1/SERCA2a vector) according to a computer-generated randomization schedule.

Efficacy/safety endpoints will be:

Echocardiogram and conductance catheterparameters

Hematology/coagulation parameters

Clinical chemistry parameters

Cardiac enzymes (CPK and troponin 1)

Inflammation markers (CRP)

Body weight

Histopathology

Analytical Plan

All treated animals will be included in the efficacy/safety analysis. For continuous variables, data distributions will be described by treatment group using means, medians, and measures of dispersion such as variances and ranges. Frequencies and proportions will be tabulated by treatment group for incidence and categorical variables. Histopathology will be descriptively examined by treatment group. Parameters measured over time will be described by treatment group for each time point and temporal trends will be assessed.

Although the present invention has been described in the context of certain preferred embodiments and examples, it is intended that the scope of the invention not be limited by that disclosure, but instead extend to the full lawful scope of the claims that follow:

We claim:

1. A method for delivering a polynucleotide to a cardiac cell in an animal, the method comprising:
substantially isolating the coronary venous circulation from the systemic circulation of the animal; and
introducing the polynucleotide into the coronary arterial circulation;
wherein the polynucleotide is substantially prevented from entering the systemic circulation.

2. The method of claim 1 wherein the polynucleotide is collected from the coronary venous circulation and recirculated to the coronary arterial circulation.

3. The method of claim 1 wherein the cardiac cell is preferentially exposed to the polynucleotide, as compared with a non-cardiac cell of the animal.

4. The method of claim 1 wherein an expression product of the polynucleotide is detectable in the cardiac cell and substantially undectectable in a non-cardiac cell of the animal.

5. The method of claim 4 wherein the expression product is detected by Western blotting.

6. The method of claim 1 wherein one or more coronary ostea are used for introduction of the polynucleotide to the coronary arterial circulation.

7. The method of claim 1 wherein an arterial delivery device is used for introduction of the polynucleotide to the coronary arterial circulation.

8. The method of claim 7 wherein the arterial delivery device is an angiographic catheter capable of engaging the coronary osteum.

9. The method of claim 1 wherein isolating the coronary venous circulation from the systemic circulation comprises occluding flow between the coronary sinus and the systemic circulation.

10. The method of claim 1 wherein the polynucleotide is collected from the coronary venous circulation using a venous collection device.

11. The method of claim 10 wherein the venous collection device is responsible for substantially occluding flow between the coronary sinus and the systemic circulation

12. The method of claim 11 wherein the venous collection device is a balloon catheter.

13. The method of claim 10 wherein the venous collection device comprises a collection lumen and a support structure adapted such that the support structure maintains patency of the coronary sinus during collection of fluid through the collection lumen.

14. The method of claim 1 wherein the method is minimally invasive.

15. The method of claim 2 wherein the polynucleotide is recirculated by an artificial flow path established between the venous collection device and the arterial delivery device.

16. The method of claim 15 wherein the artificial flow path is supplemented with flow from an auxiliary flow channel if insufficient flow of fluid at the coronary sinus is detected.

17. The method of claim 16 wherein insufficient flow is indicated by a flow rate of less than about 80 ml/min in the collection lumen.

18. The method of claim 16 wherein insufficient flow is indicated by occlusion of the coronary sinus.

19. The method of claim 16 wherein insufficient flow is indicated by occlusion of the lumen in the venous collection device.

20. The method of claim 15 wherein the artificial flow path is supplemented with flow from the auxiliary flow channel when an excessive negative pressure at a pump in the artificial flow path is detected.

21. The method of claim 20 wherein an excessive negative pressure is a pressure of less than about −140 mmHg.

22. The method of claim 2 wherein the recirculation is performed for a period equal to or less than about 2 hours.

23. The method of claim 2 wherein the recirculation is performed for a period equal to or less than about 1 hour.

24. The method of claim 2 wherein the recirculation is performed for a period of equal to or less than about 10 minutes.

25. The method of claim 2 wherein the recirculation of a solution containing the polynucleotide is performed at a flow rate of about 80-180 mL/min.

26. The method of claim 2 wherein the recirculation is performed at a pressure of about −50 mmHg at a pump in the artificial flow path.

27. The method of claim 1 wherein the heart is beating.

28. The method of claim 1, wherein the polynucleotide is capable of expressing a protein or nucleic acid molecule, the protein of nucleic molecule capable of modulating a cellular activity of the cardiac cell.

29. The method of claim 28, wherein the protein or nucleic acid molecule is capable of regulating a calcium cycling pathway of a cardiomyocyte.

30. The method of claim 28, wherein the protein is a sarcoplasmic/endoplasmic reticulum ATPase (SERCA).

31. The method of claim 30 wherein the SERCA is SERCA2a.

32. The method of claim 30 wherein where the animal has congestive heart failure the expression of SERCA in a cardiac cell of the animal is higher than that of a cardiac cell of an animal that has a similar level of congestive heart failure but has not been exposed to an exogenous polynucleotide encoding SERCA, and wherein the level of expression is measured about 1 week after exposure to the polynucleotide.

33. The method of claim 30 wherein the expression of SERCA in a cardiac cell of the animal is at least about 1.5-fold that of a cardiac cell of an animal having a similar level of chronic heart failure but has not been exposed to an exogenous polynucleotide encoding SERCA, and wherein the level of expression is measured about 1 week after exposure to the polynucleotide.

34. The method of claim 30 wherein the expression of SERCA in a cardiac cell of the animal is at least about 2-fold that of a cardiac cell of an animal having a similar level of chronic heart failure but has not been exposed to an exogenous polynucleotide encoding SERCA, and wherein the level of expression is measured about 1 week after exposure to the polynucleotide.

35. The method of claim 30 wherein where the animal has congestive heart failure the LVEF of the animal is higher than that of an animal having a similar level of congestive heart failure but has not been exposed to an exogenous polynucleotide encoding SERCA, and wherein the LVEF is measured about 1 month after exposure to the polynucleotide.

36. The method of claim 30 wherein where the animal has congestive heart failure the LVEF of the animal is about 1.6-fold that of an animal having a similar level of congestive heart failure but has not been exposed to an exogenous polynucleotide encoding SERCA, and wherein the LVEF is measured about 1 month after exposure to the polynucleotide.

37. The method of claim 28, wherein the protein is the S16E mutant of phospholamban.

38. The method of claim 28, wherein the polynucleotide is capable of expressing a molecule that is substantially antisense to a DNA or RNA sequence encoding a phospholamban protein.

39. The method of claim 28, wherein the polynucleotide is capable of expressing a small interfering RNA of phospholamban.

40. The method of claim 28 wherein the protein is a zinc finger protein capable of acting as a transcription repressor of phospholamban.

41. The method of claim 28, wherein the polynucleotide is capable of expressing an inhibitor of protein phosphatase 1.

42. The method of claim 28, wherein the protein is a channel-stabilizing protein calstabin2.

43. The method of claim 1, wherein the polynucleotide is present in a delivery vehicle.

44. The method of claim 43 wherein the delivery vehicle is a viral vector

45. The method of claim 44, wherein the viral vector is selected from the group consisting of an adenovirus, a retrovirus, a herpes simplex virus, a bovine papilloma virus, an adeno-associated virus, a lentiviral vector, a vaccinia virus, and a polyoma virus.

46. The method of claim 45, wherein the viral vector is an adenovirus vector or an adeno-associated virus vector.

47. The method of claim 45 wherein the viral vector is an AAV2/1 vector.

48. The method of claim 46 wherein the polynucleotide is inserted into the vector such that the polynucleotide is operably linked to a CAAV-based promoter.

49. The method of claim 46 wherein the polynucleotide comprises a SERCA2a coding sequence and the vector is an AAV2/1 vector.

50. The method of claim 49 wherein the sequence of the SERCA2a is inserted into the AAV2/1 vector as shown in FIG. 8.

51. The method of claim 46, wherein the number of viral particles introduced to the coronary arterial circulation, is equal to or less than about an amount selected from the group consisting of 1×10¹³, 1×10¹², 1×10¹¹, 1×10¹⁰, and 1×10⁹, and 1×10⁸ viral particles.

52. The method of claim 46, wherein the viral vector transfects at least about 50% of cells of a targeted cardiac tissue and wherein the transfection level is measured about 1 week after exposure to the polynucleotide.

53. The method of claim 46, wherein the viral vector transfects at least about 80% of cells of a targeted cardiac tissue and wherein the transfection level is measured about 1 week after exposure to the polynucleotide.

54. The method of claim 49, wherein delivery of the SERCA2a improves the ejection fraction of a heart of the animal by at least about 20% and wherein the ejection fraction is measured about 1 month after exposure to the polynucleotide.

55. The method of claim 28, wherein the modulation of the cellular activity of the cardiac cell is sustained for at least about one month.

56. The method of claim 1, wherein the delivery of said polynucleotide prevents or treats a disease of the cardiovascular system.

57. The method of claim 56, wherein the disease is selected from the group consisting of heart failure, ischemia, arrhythmia, myocardial infarction, congestive heart failure, transplant rejection, abnormal heart contractility, non-ischemic cardiomyopathy, mitral valve regurgitation, aortic stenosis or regurgitation, and abnormal Ca²⁺ metabolism.

58. The method of claim 1, wherein the delivery of the polynucleotide sustains the ejection fraction of a heart of said animal at a level approximately equal to the level of the heart before delivery of the polynucleotide.
